(19) United States
(12) Patent Application Publication
(10) Pub. No.: US 2007/0093743 A1
(43) Pub. Date: Apr. 26, 2007

(54) Iontophoresis Drug Delivery Device Providing Acceptable Depth and Duration of Dermal Anesthesia

(75) Inventors: Preston Keusch, Jonesboro, ME (US); Vilambi NRK Reddy, Tamil Nadu (IN); Ashutosh Sharma, Springfield, NJ (US); George M. Baskinger, North Haledon, NJ (US)

Correspondence Address: KIRKPATRICK & LOCKHART PRESTON GATES ELLIS LLP, 535 SMITHFIELD STREET, PITTSBURGH, PA, 15222, US

(73) Assignee: Vyteris, Inc., Fair Lawn, NJ (US)

(21) Appl. No.: 11/535,608

(22) Filed: Sep. 27, 2006

Related U.S. Application Data
(60) Provisional application No. 60/722,640, filed on Sep. 30, 2005.

(51) Int. Cl. A61N 1/30

(52) U.S. Cl. 604/20

(57) ABSTRACT

An electrically assisted transdermal drug delivery system for highly effective electrotransport of an anesthetic and a vasoconstrictor producing clinically acceptable depth and duration of dermal anesthesia at a treatment site. In certain embodiments, the anesthetic comprises lidocaine and the vasoconstrictor comprises epinephrine.

### CROSS REFERENCE TO RELATED APPLICATION

The present application claims priority under 35 U.S.C. § 119(e) from co-pending U.S. Patent Application Ser. No. 60/722,640, filed Sep. 30, 2005.

### BACKGROUND

1. Field of the Invention

The present disclosure relates to highly shelf-stable electrically assisted transdermal drug delivery systems for producing acceptable depth and duration of dermal anesthesia, and uses therefor.

2. Description of the Related Art

Transdermal drug delivery systems have become an increasingly important means of administering drugs. Such systems offer advantages clearly not achievable by other modes of administration such as avoiding introduction of the drug through the gastrointestinal tract or punctures in the skin, to name a few.

There are two types of transdermal drug delivery systems: “passive” and “active”. Passive systems deliver drug through the skin of the user unaided, an example of which would involve the application of a topical anesthetic to provide localized relief, as disclosed in U.S. Pat. No. 3,814,095. Active systems, on the other hand, use external force to facilitate delivery of a drug through a patient's skin. Examples of active systems include ultrasound, electroporation, electroosmosis, and/or iontophoresis.

Iontophoretic delivery of a medicament is accomplished by application of a voltage to a medicament-loaded reservoir-electrode, sufficient to maintain a current between the medicament-loaded reservoir-electrode and a return electrode (another electrode) applied to a patient's skin so that an ionic form of the desired medicament is delivered to the patient.

Conventional iontophoretic devices, such as those described in U.S. Patent Nos. 4,820,263, 4,927,408, and 5,084,008, the disclosures of which are hereby incorporated by reference, deliver a drug transdermally by iontophoresis. These devices typically contain two electrodes—an anode and a cathode. In a typical iontophoretic device, electric current is driven from an external power supply. In a device for delivering drug from an anode, positively charged drug is delivered into the skin at the anode, with the cathode completing the electrical circuit. Likewise, in a system for delivering drug from a cathode, negatively charged drug is delivered into the skin at the cathode, with the anode completing the electrical circuit. Accordingly, there has been considerable interest in iontophoresis to perform delivery of drugs for a variety of purposes. One example is the delivery of lidocaine, a common topical, local anesthetic.

A number of iontophoretic systems have been described for delivery of lidocaine in order to affect dermal anesthesia. Concomitant delivery of both lidocaine and a vasoconstrictor, such as epinephrine has been found to be most desirable. The vasoconstrictor retards the migration of the lidocaine from the site of delivery.

International Patent Publication WO 98/208869 discloses an iontophoretic device for delivery of epinephrine HCl and lidocaine HCl. Likewise, U.S. Pat. Nos. 4,786,277 and 6,295,469, and EP 0941 085 B1 disclose iontophoresis devices for delivery of lidocaine. Commercial products exist for delivery of lidocaine and epinephrine. For example, Iomed, Inc. markets an iontophoresis system sold under the trademark Numby Stuff® for local delivery of lidocaine and epinephrine by iontophoresis. That device is marketed as a kit containing active and return electrode pairs and a controller. A multiple-use vial of a solution of 2% lidocaine HCl and 1:100,000 epinephrine (referred to by the trademark Iontocaine®) is included with the kit. The system has to be assembled and the liquid containing lidocaine and epinephrine is then added to the active patch just before use. This system has several disadvantages. A practitioner may lose track of the age of the multi-use vial of lidocaine and epinephrine, consequently allowing the epinephrine to degrade in the vial. It also is cumbersome to preload a patch just before use. A syringe is used for loading the patch during each use and the potential for dose-to-dose variation is present. For example, the loading syringe may not be filled with the proper amount of solution, a portion of the solution may not be applied to the patch, and/or the liquid can separate out of the absorbent drug-containing electrode because the solution is a separate phase from the absorbent reservoir.

One technical hurdle in the iontophoretic delivery of lidocaine and epinephrine is achieving the combination of rapid and effective dermal anesthesia producing acceptable depth and duration without causing patient discomfort. Lidocaine and epinephrine formulations are known in the art that deliver up to 2% lidocaine with very low amounts of epinephrine in the ratio of 1:100,000. To achieve rapid and effective dermal anesthesia of acceptable depth, the applied current in such a device either needs to be uncomfortably high, or the lower current must be applied for relatively long periods of time (i.e., the charge density must be high). Also, for long-duration effects, epinephrine levels need to be high. High charge densities (traditionally in the range of greater than 2.0 milliAmp·minutes/centimeters² (mA·min/cm²)) and high drug concentrations are known to cause discomfort during delivery and often result in such combined side effects as erythema and edema after drug delivery. It is believed that, to date, there are no teachings on how to make a high-current iontophoretic device for comfortably delivering efficacious doses of lidocaine and epinephrine in a reasonably short time, while producing acceptable depth and duration of dermal anesthesia at the treated site.

### SUMMARY OF INVENTION

Provided is an iontophoretic device for delivery of a topical anesthetic, such as lidocaine, in combination with a vasoconstrictor, such as epinephrine, providing acceptable onset, depth and duration of dermal anesthesia at the treated site.

In certain embodiments of the device, the drug is stored as a solid solution in a solid solution reservoir thereby avoiding squeezing out of drug and changes in the active area of the reservoir. The device includes an electrode and a hydrophilic polymeric reservoir situated in electrically conductive relation to the electrode and, in certain non-limiting embodiments is ready for use immediately upon removal from its packaging. In other words, in such embodiments there is no need to load the active ingredients in the anode or the return solution in the cathode just prior to use. Certain embodiments of the device are pharmaceutically, chemically, electrochemically, and physically stable for more than 24 months at room-temperature, with stability for extended periods at elevated temperatures, making manufacture, distribution and storage more effective and providing the end user a greater confidence in the product, with less returns of the device from customer. Further, certain non-limiting embodiments of the iontophoretic device provide acceptable depth and duration of dermal anesthesia after a short delivery time of less than about ten minutes at high current fluxes, with minimal unpleasant or painful sensation during delivery, and with clinically acceptable reversible erythema and edema skin effects.

Accordingly, an aspect of the present disclosure is directed to an iontophoresis electrode assembly comprising an anode assembly including a pre-loaded hydrogel drug reservoir in electrical contact with a first electrode, the drug reservoir comprising a drug formulation including an anesthetic and a vasoconstrictor, and wherein the electrode assembly produces clinically acceptable depth and duration of dermal anesthesia at a treated skin site on a patient.

In certain non-limiting embodiments of an iontophoresis electrode assembly according to the present disclosure, the anesthetic comprises lidocaine and/or the vasoconstrictor comprises epinephrine. In certain non-limiting embodiments of an iontophoresis electrode assembly according to the present disclosure, the drug reservoir includes lidocaine in an amount greater than about 2% by weight of the reservoir, preferably in an amount of about 2% to about 12% by weight of the reservoir, and more preferably in an amount of about 10% by weight of the reservoir. In certain non-limiting embodiments of an iontophoresis electrode assembly according to the present disclosure, the drug reservoir includes epinephrine in an amount greater than about 0.005% by weight of the reservoir, preferably in an amount greater than about 0.01% by weight of the reservoir, and more preferably in an amount of about 0.01% to about 0.3% by weight of the reservoir. In certain non-limiting embodiments, the reservoir includes about 2% to about 12% lidocaine by weight of the reservoir, and about 0.001% to about 0.3% epinephrine by weight of the reservoir.

An additional aspect of the present disclosure is directed to an iontophoresis electrode assembly comprising an anode assembly including a pre-loaded hydrogel drug reservoir in electrical contact with a first electrode, the drug reservoir comprising a drug formulation including an anesthetic and a vasoconstrictor, and wherein the average depth to which all sensation is eliminated on advancing an 18 gauge needle into the treated skin of a forearm of a patient immediately after treatment with the electrode assembly and the drug formulation is greater than 4 mm, more preferably greater than 5 mm, and even more preferably at least 6 mm.

A further aspect of the present disclosure is directed to an iontophoresis electrode assembly comprising an anode assembly including a pre-loaded hydrogel drug reservoir in electrical contact with a first electrode, the drug reservoir comprising a drug formulation including an anesthetic and a vasoconstrictor, and wherein the average depth to which all sensation is eliminated on advancing an 18 gauge needle into the treated skin of a forearm of a patient immediately after treatment with the electrode assembly and the drug formulation is statistically significantly greater (p<0.0001) than the average depth to which sensation of pain is eliminated assessed in an identical manner on a substantially equivalent skin site immediately after treatment using an identical electrode assembly iontophoretically delivering a placebo.

Yet an additional aspect of the present disclosure is directed to an iontophoresis electrode assembly comprising an anode assembly including a pre-loaded hydrogel drug reservoir in electrical contact with a first electrode, the drug reservoir comprising a drug formulation including an anesthetic and a vasoconstrictor, and wherein the average depth to which all sensation is eliminated on advancing an 18 gauge needle into the treated skin of a forearm of a patient immediately after treatment with the electrode assembly and the drug formulation is at least 2 mm greater than the average depth to which all sensation is eliminated assessed in an identical manner on a substantially equivalent skin site immediately after treatment using an identical electrode assembly iontophoretically delivering a placebo.

Yet a further aspect of the present disclosure is directed to an iontophoresis electrode assembly comprising an anode assembly including a pre-loaded hydrogel drug reservoir in electrical contact with a first electrode, the drug reservoir comprising a drug formulation including an anesthetic and a vasoconstrictor, and wherein the average depth to which all sensation is eliminated on advancing an 18 gauge needle into the treated skin of a forearm of a patient after treatment with the electrode assembly and the drug formulation does not decrease within the first hour immediately after ending the treatment.

Yet another aspect of the present disclosure is directed to an iontophoresis electrode assembly comprising an anode assembly including a pre-loaded hydrogel drug reservoir in electrical contact with a first electrode, the drug reservoir comprising a drug formulation including an anesthetic and a vasoconstrictor, and wherein the average pain threshold depth on advancing an 18 gauge needle into the treated skin of a forearm of a patient immediately after treatment with the electrode assembly and the drug formulation is greater than 2 mm, and more preferably is at least 3 mm.

An additional aspect of the present disclosure is directed to an iontophoresis electrode assembly comprising an anode assembly including a pre-loaded hydrogel drug reservoir in electrical contact with a first electrode, the drug reservoir comprising a drug formulation including an anesthetic and a vasoconstrictor, and wherein the average pain threshold depth on advancing an 18 gauge needle into the treated skin of a forearm of a patient immediately after treatment with the electrode assembly and the drug formulation is statistically significantly greater (p<0.0001) than the average pain threshold depth assessed in an identical manner on a substantially equivalent skin site immediately after treatment using an identical electrode assembly iontophoretically delivering a placebo.

A further aspect of the present disclosure is directed to an iontophoresis electrode assembly comprising an anode assembly including a pre-loaded hydrogel drug reservoir in electrical contact with a first electrode, the drug reservoir comprising a drug formulation including an anesthetic and a vasoconstrictor, and wherein the average pain threshold depth on advancing an 18 gauge needle into the treated skin of a forearm of a patient immediately after treatment with the electrode assembly and the drug formulation is at least 2 mm greater, and preferably is at least 3 mm greater, than the average pain threshold depth assessed in an identical manner on a substantially equivalent skin site immediately after treatment using an identical electrode assembly iontophoretically delivering a placebo.

Another aspect of the present disclosure is directed to an iontophoresis electrode assembly comprising an anode assembly including a pre-loaded hydrogel drug reservoir in electrical contact with a first electrode, the drug reservoir comprising a drug formulation including an anesthetic and a vasoconstrictor, and wherein the average pain threshold on advancing an 18 gauge needle into the treated skin of a forearm of a patient after treatment with the electrode assembly and the drug formulation does not decrease within the first hour immediately after ending the treatment.

Yet additional aspects of the present disclosure are directed to methods using any of the foregoing iontophoresis electrode assemblies to produce local anesthesia in a patient and provide the aforementioned results.

Certain non-limiting embodiments within the scope of the present disclosure are directed to methods of producing local anesthesia in a patient including applying a charge density of at least about 1.5 mA·min/cm² for at least about 5 minutes to an electrically assisted drug delivery system comprising an anode assembly including a reservoir in electrical contact with the patient, wherein the reservoir is pre-loaded with a drug formulation including an anesthetic and a vasoconstrictor, and wherein the electrically assisted drug delivery system produces clinically acceptable depth and duration of dermal anesthesia at a treated site. In certain non-limiting embodiments of the method, the current density is between about 1.5 mA·min/cm² and about 4.2 mA·min/cm², and may be about 3.4 mA·min/cm². Also, in certain non-limiting embodiments of the method, the charge density is applied for a time period of from about 5 to about 20 minutes.

### BRIEF DESCRIPTION OF THE DRAWINGS

FIG. 1 (prior art) shows schematically an electrically assisted drug delivery system including an anode assembly, a cathode assembly, and a controller/power supply.

FIG. 2 is an exploded isometric view of various aspects of one embodiment of an integrated electrode assembly provided in accordance with the present invention.

FIG. 3 is an exploded isometric view of various aspects of one embodiment of an integrated electrode assembly provided in accordance with the present invention.

FIG. 4 is an elevational view of various aspects of one embodiment of an integrated electrode assembly provided in accordance with the present invention.

FIG. 5A is an exploded isometric view illustrating various aspects of the interconnection between a portion of an integrated electrode assembly of one embodiment of an electrically assisted drug delivery device provided in accordance with the present invention with other components of the electrically assisted delivery device.

FIG. 5B is a schematic representation of the interaction between a portion of an integrated electrode assembly of one embodiment of an electrically assisted drug delivery device provided in accordance with the present invention and other components of the electrically assisted drug delivery device.

FIG. 5C is a schematic representation of the interaction between a portion of an integrated electrode assembly of one embodiment of an electrically assisted drug delivery device provided in accordance with the present invention and other components of the electrically assisted drug delivery device.

FIG. 6 is a schematic elevational view of various aspects of an integrated electrode assembly provided in accordance with the present invention.

FIGS. 6B and 6C are cross-sectional views illustrating aspects of the electrode assembly of FIG. 6.

FIG. 7 is a schematic elevational view of various aspects of an integrated electrode assembly provided in accordance with the present invention.

FIG. 7A is a cross-sectional view of the release cover of FIG. 7.

FIG. 8 is a schematic that illustrates the effect of electrode geometry and spacing on the delivery paths of a composition through a membrane.

FIG. 9 is a schematic that illustrates the effect of electrode geometry and spacing on the delivery paths of a composition through a membrane.

FIG. 10 is a cross-sectional view of one embodiment of an un-loaded electrode assembly in contact with a loading solution.

FIG. 11 is a cut-away view of a one embodiment of a package including an embodiment of an electrode assembly structured in accordance with the present invention.

FIGS. 12A and 12B are plots of mean pain threshold over time, and change in sensory depth over time reflecting study data described herein

FIG. 13 is a plot of average CP over time reflecting data generated in study described herein involving active and placebo iontophoresis treatments.

FIG. 14 is a plot of erythema indices over time reflecting data generated in a study described herein involving active and placebo iontophoresis treatments.

### DETAILED DESCRIPTION

### Definitions

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. Also, the disclosure of these ranges is intended as a continuous range including every value between the minimum and maximum values.

Unless otherwise specified, embodiments of the present invention are employed under “normal use” conditions, which refer to use within standard operating parameters for those embodiments. During operation of various embodiments described herein, a deviation from a target value of one or more parameters of about ±10% or less for an iontophoretic device under “normal use” is considered an adequate deviation for purposes of the present invention.

Described herein are embodiments of an electrode assembly for electrically assisted transmembrane delivery of drugs, for example lidocaine and epinephrine, with acceptable depth and duration of dermal anesthesia at the treated site. Certain embodiments of the electrode assembly exhibit exceptional shelf-stability, even at temperatures greater than room temperature (25° C.).

The terms “unloaded” or “unloaded reservoir,” are necessarily defined by the process of loading a reservoir. In the loading process, a drug or other compound or composition is absorbed, adsorbed, and/or diffused into a reservoir to reach a final content or concentration of the compound or composition. An unloaded reservoir is a reservoir that lacks that compound or composition in its final content or concentration. In one example, the unloaded drug reservoir is a hydrogel, as described in further detail below, that includes water and a salt. One or more additional ingredients may be included in the unloaded reservoir. Typically, active ingredients are not present in the unloaded gel reservoir. Other additional, typically non-ionic ingredients, such as preservatives, may be included in the unloaded reservoir. Although the salt may be one of many salts, including alkaline metal halide salts, the salt typically is sodium chloride (NaCl). Other halide salts such as, without limitation, potassium chloride (KCl) or lithium chloride (LiCl) might be substantially equivalent to NaCl in terms of functionality, but may not be preferred. Use of halide salts to prevent electrode corrosion is disclosed in U.S. Pat. Nos. 6,629,968 and 6,635,045, both of which are incorporated herein by reference in their entireties.

The terms “electrically assisted delivery” or “electrically assisted drug delivery” refer to the facilitation of the transfer of any compound across a membrane, such as, without limitation, skin, mucous membranes, and nails, by the application of an electric potential across that membrane. These terms are intended to include, without limitation, iontophoretic, electrophoretic, and electroendosmotic delivery methods. By “active ingredient” it is meant, without limitation, drugs, active agents, therapeutic compounds, and any other compound capable of eliciting any pharmacological effect in the recipient that is capable of transfer by electrically assisted delivery methods. A “transdermal device” or “transdermal patch” includes both active and passive transdermal devices and patches.

The term “lidocaine,” unless otherwise specified, refers to any water-soluble form of lidocaine in substantially ionic form, including salts or derivatives, homologs, or analogs thereof. For example, as is used in Examples below, “lidocaine” refers to lidocaine hydrochloride (HCl), in substantially ionic form, commercially available, for example, as XYLOCAINE® (a trademark of AstraZeneca LP of Wayne, Pa.), among other names.

The term “epinephrine” refers to any form of epinephrine, salts, its free base or derivatives, homologs, or analogs thereof, so long as they can be solubilized in an aqueous solution, and is present in substantially ionic form. For example, as is used in the examples below, “epinephrine” refers to epinephrine bitartrate, in substantially ionic form.

As applied to various embodiments of electrically assisted delivery devices described herein, the term “integrated” as used in connection with a device indicates that at least two electrodes are associated with a common structural element of the device. For example, and without limitation, a transdermal patch of an iontophoretic device may include both a cathode and an anode “integrated” therein, i.e., the cathode and anode are attached to a common backing.

As applied to various embodiments of electrically assisted delivery devices described herein, a “flexible” material or structural component is generally compliant and conformable to a variety of membrane surface area configurations, and a “stiff” material or structural component is generally not compliant and not conformable to a variety of membrane surface area configurations. In addition, a “flexible” material or component possesses a lower flexural rigidity in comparison to a “stiff” material or structural component having a higher flexural rigidity. For example, and without limitation, a “flexible” material when used as a backing for an integrated patch can substantially conform over the shape of a patient's forearm or inside elbow, whereas a comparatively “stiff” material would not substantially conform over such shapes in the same use as a backing.

As applied herein, the term “transfer absorbent” includes any medium structured to retain therein a fluid or fluids on an at least temporary basis and to release the retained fluids to another medium such as a hydrogel reservoir, for example. Examples of “transfer absorbents” that may be employed herein include, without limitation, non-woven fabrics and open-cell foams and sponges.

As used herein, “stable” and “stability” refer to a property of individual packaged electrode-reservoir assemblies, and typically is demonstrated statistically. The term “stable” refers to retention of a desired quality, with particular, but not exclusive focus on active ingredients such as epinephrine content, lidocaine content, hydrogel strength, hydrogel tack, electrochemical capacity and electronic conductivity, within a desired range. For example, in an iontophoretic device, the U.S. Food and Drug Administration (FDA) may require retention, as a lot, of 90% of the label claim of epinephrine over a given time period using a least square linear regression statistical method with a 95% confidence level. As used herein, however, an electrode assembly and/or parts thereof are considered stable so long as they substantially retain their desired function in an iontophoretic system. Stability, though measured by any applicable statistical method, is a quality of the electrode assembly. Therefore, methods other than FDA-approved statistical methods may be used to quantitatively assess stability. For instance, even though for FDA purposes a 95% confidence level may be required, those limits are not literally required for a device to be called “stable.” Similarly, and for exemplary purposes only, a “stable” iontophoretic electrode may be said to retain 80% of the original epinephrine concentration over a given time period, as determined by least square linear regression analysis.

As used generally herein, certain non-limiting embodiments of an electrode-reservoir, reservoir, or electrode assembly according to the present disclosure are stable when hermetically sealed for a given time period. This means that when the electrode assembly is sealed in a container that is impermeable to oxygen and water (“hermetically sealed”), the electrode-reservoir retains a specified characteristic or parameter within desired boundaries for a given time period. By “original concentration”, “original amounts”, or “original levels” it is meant the concentration, amount, or level of any constituent or physical, electrochemical, or electrical parameter relating to the electrode assembly at a time point designated as t=0, and typically refers to a time point after the electrode assembly is sealed within the hermetically sealed container. This time may take up to a few weeks to ensure uniform distribution of ingredients in the reservoir(s).

As used herein, “anesthesia” refers to a state characterized by a loss of sensation as a result of pharmacologic depression of nerve function.

As used herein, “non-necrotizing” refers to not causing necrosis, wherein necrosis is defined as death of tissues or cells caused when not enough blood is supplied to the tissues or cells due to injury. With particular reference to “non-necrotizing amount of vasoconstrictor,” the amount of vasoconstrictor delivered in the invention does not cause the tissue in contact with the vasoconstrictor to be injured to the point wherein blood supply is substantially compromised, causing cellular death.

Iontophoretic Device

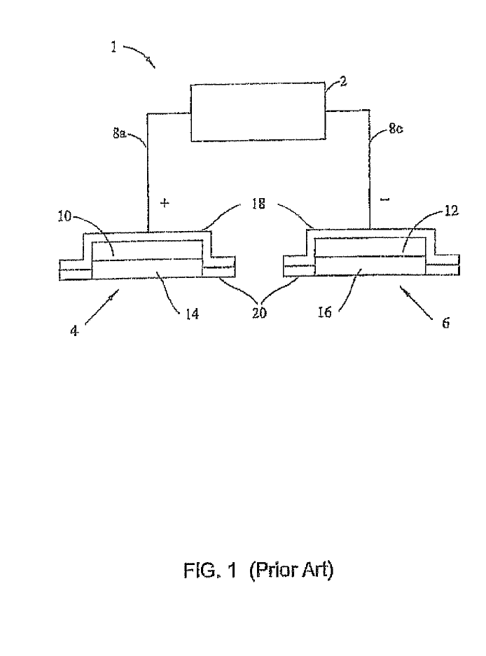

FIG. 1 depicts schematically a typical electrically assisted drug delivery apparatus 1. The apparatus 1 includes an electrical power supply/controller 2, an anode electrode assembly 4, and a cathode electrode assembly 6. Anode electrode assembly 4 and cathode electrode assembly 6 are connected electrically to the power supply/controller 2 by conductive leads 8a and 8c, respectively. The anode electrode assembly 4 includes an anode 10 and the cathode electrode assembly 6 includes a cathode 12. The anode 10 and the cathode 12 are both in electrical contact with the leads 8a, 8c. The anode electrode assembly 4 further includes an anode reservoir 14, while the cathode electrode assembly 6 further includes a cathode reservoir 16. Both the anode electrode assembly 4 and the cathode electrode assembly 6 include a backing 18 to which a pressure sensitive adhesive 20 is applied in order to affix the electrode assemblies 4, 6 to a membrane (e.g., skin of a patient), to establish electrical contact for the reservoirs 14, 16 with the membrane. Optionally, the reservoirs 14, 16 may be at least partially covered with the pressure sensitive adhesive 20.

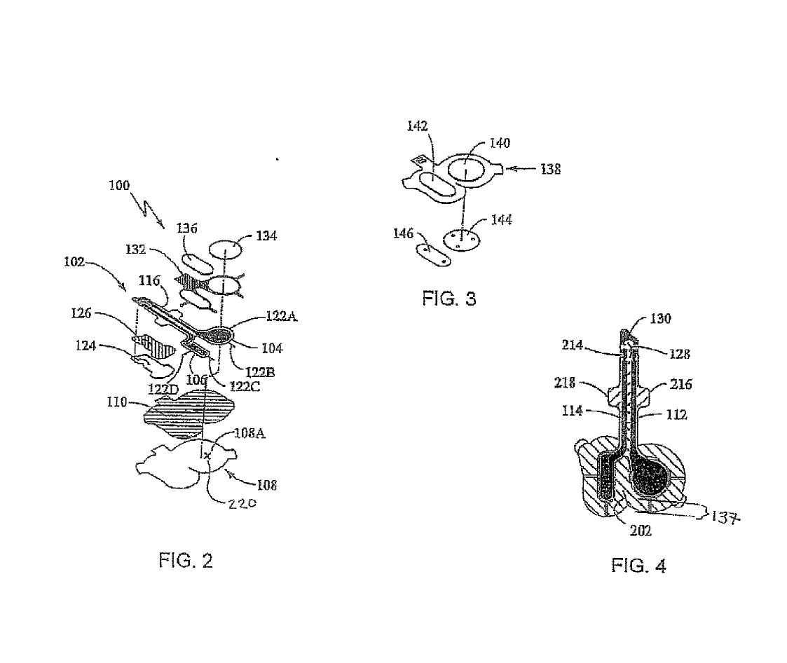

FIGS. 2 through 10 illustrate various aspects of one non-limiting embodiment of an integrated electrode assembly 100 of the present disclosure structured for use with an electrically assisted delivery device, for example, for delivery of a composition through a membrane. A printed electrode layer 102 including two electrodes (an anode 104 and a cathode 106) is connected to a flexible backing 108 by a layer of flexible backing adhesive 110 positioned between the printed electrode layer 102 and the flexible backing 108. One or more leads 112, 114 may extend from the anode 104 and/or cathode 106 to a tab end portion 116 of the printed electrode layer 102. In various aspects, an insulating dielectric coating 118 may be deposited on and/or adjacent to at least a portion of one or more of the electrodes 104, 106 and/or the leads 112, 114. The dielectric coating 118 may serve to strengthen or bolster the physical integrity of the printed electrode layer 102; to reduce point source concentrations of current passing through the leads 112, 114 and/or the electrodes 104, 106; and/or to resist creating an undesired short circuit path between portions of the anode 104 and its associated lead 112 and portions of the cathode 106 and its associated lead 114.

In other aspects, one or more splines 122A, 122B, 122C, 122D may be formed to extend from various portions of the printed electrode layer 102, as shown. It can be seen that at least one advantage of the splines 122 is to facilitate manufacturability (e.g., die-cutting of the electrode layer 102) and construction of the printed electrode layer 102 for use in the assembly 100. The splines 122 may also help to resist undesired vacuum formation when a release cover (see discussion hereafter) is positioned in connection with the electrode layer 102 during construction or use of the assembly 100.

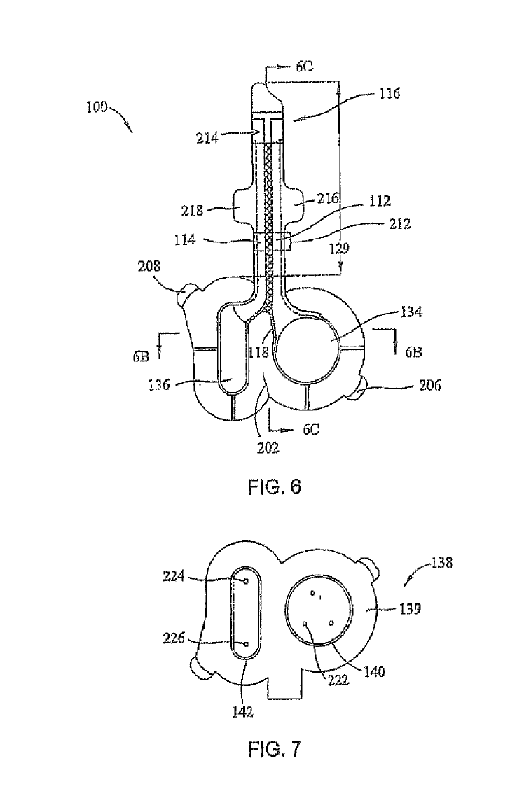

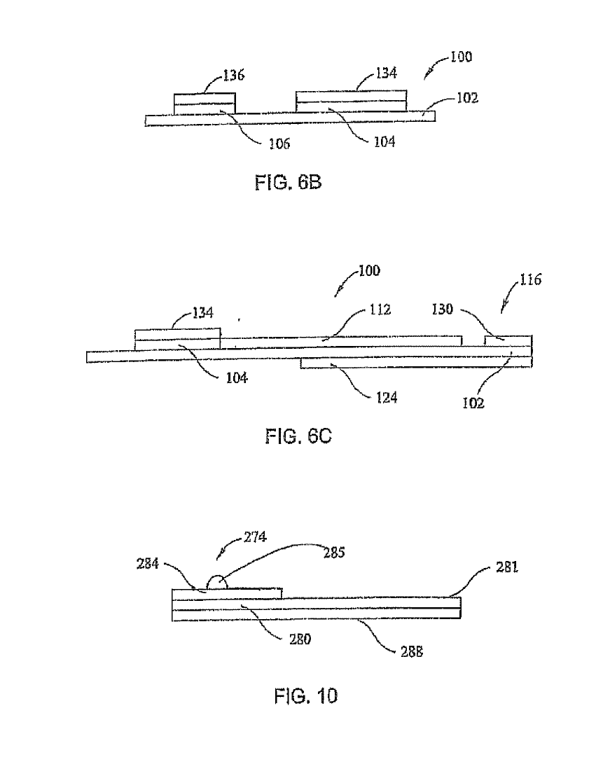

In certain non-limiting embodiments of the present invention, a tab stiffener 124 is connected to the tab end portion 116 of the printed electrode layer 102 by a layer of adhesive 126 positioned between the tab stiffener 124 and the tab end portion 116. In various embodiments, a tab slit 128 may be formed in the tab end portion 116 of the assembly 100 (as shown more particularly in FIGS. 2 and 4). The tab slit 128 may be formed to extend through the tab stiffener 124 and the layer of adhesive 126. In other embodiments, a minimum tab length 129 (as shown particularly in FIG. 6) for the depicted embodiment as structured in association with the tab end portion 116 may be in the range of at least about 1.5 inches.

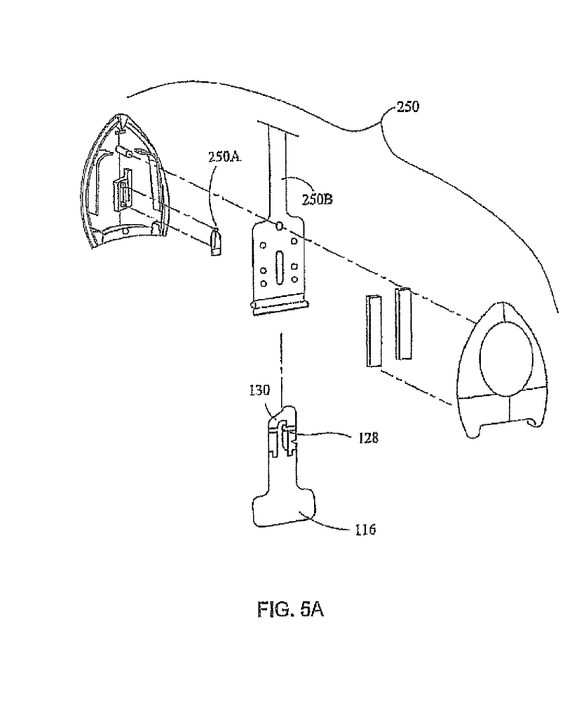

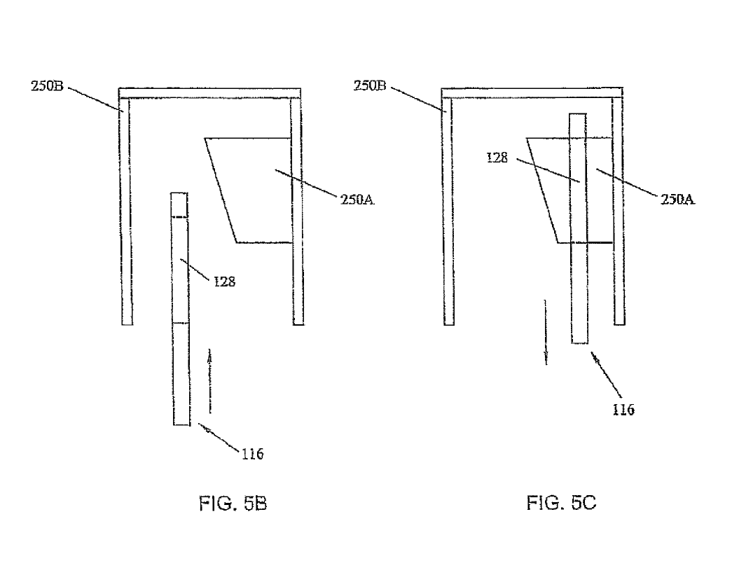

With reference to FIGS. 6A-5C, the tab end portion 116 may be structured to be mechanically or electrically operatively associated with one or more other components of an electrically assisted drug delivery device such as a knife edge 250A of a connector assembly 250, for example. As shown schematically in FIGS. 5B and 5C, once the tab end portion 116 is inserted into a flexible circuit connector 250B of the connector assembly 250, the tab slit 128 of the tab end portion 116 may be structured to receive therein the knife edge 250A. It can be appreciated that the interaction between the knife edge 250A and the tab slit 128 may serve as a tactile sensation aid for a user manually inserting the tab end portion 116 into the flexible circuit connector 250B of the connector assembly 250. In addition, the knife edge 250A may be structured, upon removal of the tab end portion 116 from the connector assembly 250, to cut or otherwise disable one or more electrical contact portions positioned on the tab end portion 116, such as a sensor trace 130, for example. It can be seen that this disablement of the electrical contact portions may reduce the likelihood that unintended future uses of the assembly 100 will occur after an initial use of the assembly 100 and the connector assembly 250 for delivery of a composition to a membrane, for example.

In other aspects, a layer of transfer adhesive 110 may be positioned in communication with the printed electrode layer 102 to facilitate adherence and/or removal of the assembly 100 from a membrane, for example, during operation of an electrically assisted delivery device that includes the assembly 100. Optionally, a second adhesive layer 132 may be positioned on the electrode layer to peripherally surround the printed electrode layer 102 and to further facilitate adherence and/or removal of the assembly 100 from a membrane. As shown in FIG. 2, a first hydrogel reservoir 134 is positioned for electrical communication with the anode 104 of the printed electrode layer 102 and a second hydrogel reservoir 136 is positioned for electrical communication with the cathode 106 of the printed electrode layer 102. In other aspects, although a hydrogel may be preferred in many instances, there may be substantially no hydrogel reservoir associated with the cathode 106, or a non-hydrogel substance including NaCl, for example, may be associated with the cathode 106.

As shown in FIG. 3, a release cover 138 includes an anode-donor absorbent well 140 and a cathode-return absorbent well 142. The anode-donor absorbent well 140 is structured to receive therein a donor transfer absorbent 144 suitably configured/sized for placement within the anode-donor absorbent well 140. Likewise, the cathode-return absorbent well 142 is structured to receive therein a return transfer absorbent 146 suitably configured/sized for placement within the cathode-return absorbent well 142. The transfer absorbents 144, 146 may be attached to their respective wells 140, 142 by a suitable method or apparatus, such as by use of one or more spot welds, for example. In construction of the assembly 100, it can be seen that the release cover 138 is structured for communication with the flexible backing adhesive layer 110 such that the donor transfer absorbent 144 establishes contact with the hydrogel reservoir 134 associated with the anode 104 and the return transfer absorbent 146 establishes contact with the hydrogel reservoir 136 associated with the cathode 106.

In various embodiments, the integrated assembly 100 may include a first reservoir-electrode assembly (including the reservoir 134 and the anode 104) charged with lidocaine HCl and epinephrine bitartrate, for example, that may function as a donor assembly, and a second reservoir-electrode assembly (including the reservoir 136 and the cathode 106) that may function as a return assembly. The assembly 100 includes the reservoir-electrode 104 and the reservoir-electrode 106 mounted on an electrode assembly securement surface 108A of the flexible backing 108. As described above, the assembly 100 includes two electrodes, an anode 104 and a cathode 106, each having an electrode surface and an operatively associated electrode trace or lead 112 and 114, respectively. The electrodes 104, 106 and the electrode traces 112, 114 may be formed as a thin film deposited onto the electrode layer 102 by use of a conductive ink, for example. The conductive ink may include Ag and Ag/AgCl, for example, in a suitable binder material, and the conductive ink may have the same composition for both the electrodes 104, 106 and the electrode traces 112, 114. A substrate thickness for the conductive ink may be, for example, in the range of about 0.002 inches to 0.007 inches. In other aspects of the embodiment, the specific capacity of the conductive ink, for Ag/AgCl electrochemistry, is preferably in the range of about 2 to 120 mA·min/cm², or more preferably in the range of 5 to 20 mA·min/cm². In various aspects, the conductive ink may comprise a printed conductive ink. The electrodes 104, 106 and the electrode traces 112, 114 may be formed on the electrode layer 102 to provide a relatively stiff portion of the assembly 100.

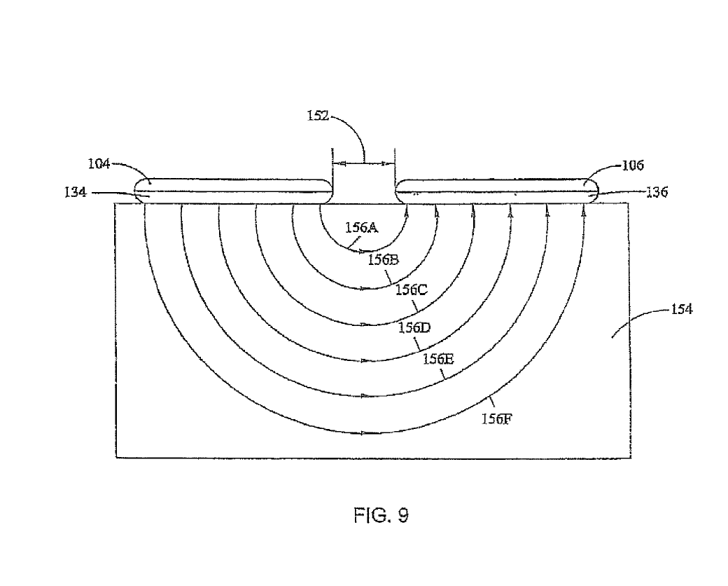

In various embodiments of the present invention, a shortest distance 152 between a surface area of the anode 104/reservoir 134 assembly and a surface area of the cathode 106/reservoir 136 assembly may be in the range of at least about 0.635 cm (0.25 inches). Referring now to FIG. 8, for example, it can be seen that inappropriate selection of the distance 152, the geometric configuration of the electrodes 104, 106 (e.g., thickness, width, total surface area, and others), and/or a combination of other factors may result in a substantially non-uniform delivery of a composition between the electrodes through a membrane 154 during operation of the assembly 100. As shown, the delivery of the composition through the membrane is shown schematically by composition delivery paths 156A-156F. In contrast, as shown in FIG. 9, appropriate selection of the distance 152, the geometric configuration of the electrodes 104, 106 (for example, thickness, width, and total surface area), and/or a combination of other factors may result in a substantially uniform delivery of a composition between the electrodes through a membrane 154 as shown by delivery paths 156A-156F. It can be seen that the inventors have recognized the problem of delivering a composition through a membrane that may include scar tissue, for example, or another variation in the density of the membrane that may adversely impact the effectiveness and uniformity of delivery of the composition between the electrodes of a device, for example.

In accordance with the discussion above, the electrodes 104, 106 may each be mounted with bibulous reservoirs 134, 136, respectively, formed from a cross-linked polymeric material such as cross-linked poly(vinylpyrrolidone) (“PVP”) hydrogel, for example, including a substantially uniform concentration of a salt, for example. The reservoirs 134, 136 may also include one or more reinforcements, such as a low basis weight non-woven scrim, for example, to provide shape retention to the hydrogels. The reservoirs 134, 136 each may have adhesive and cohesive properties that provide for releasable adherence to an applied area of a membrane (e.g., the skin of a patient). In various embodiments, the strength of an adhesive bond formed between portions of the assembly 100 and the application area or areas of the membrane is less than the strength of an adhesive bond formed between the membrane and the reservoirs 134, 136. These adhesive and cohesive properties of the reservoirs 134, 136 have the effect that when the assembly 100 is removed from an applied area of a membrane, a substantial amount of adhesive residue, for example, does not remain on the membrane. These properties also permit the reservoirs 134, 136 to remain substantially in electrical communication with their respective electrodes 104, 136 and the flexible backing 108 to remain substantially in communication with the printed electrode layer 102.

Portions of the assembly 100 as provided in accordance with certain embodiments of the present invention, may be structured to exhibit flexibility or low flexural rigidity in multiple directions along the structure of the device 100. Working against flexibility of the device 100, however, may be the construction of the comparatively stiffer electrode layer 102, which may include a material such as print-treated polyethylene terephthalate (“PET”), for example, as a substrate. PET is a relatively strong material exhibiting high tensile strength in both the machine and transverse directions and having a flexural rigidity, G=E*δⁿ, which is a function of modulus of elasticity (E) and a power of the thickness (δ) of the material. By way of a hypothetical counter-example, if a substance such as Mylar™, for example, were to be used for both the electrode layer 102 and the flexible backing 108, at least two problems could be presented: (1) the assembly 100 may be too inflexible to fully or effectively adhere to a site of treatment on a membrane, and (2) upon removal from the membrane once treatment is completed, the assembly 100 would require a relatively high level of force, due to the strength of the flexible backing 108, to remove the assembly 100.

Certain embodiments of the present invention provide the flexible backing 108 around the periphery of the stiff electrode layer 102. In certain aspects of particular embodiments, a relatively thin and highly compliant flexible backing composed of about 0.004 inch ethylene vinyl acetate (“EVA”), for example, may be used for the flexible backing 108. This configuration offers a flexible and compliant assembly 100 in multiple planar directions, permitting the assembly 100 to conform to the contour of a variety of membranes and surfaces. In addition, a pressure sensitive adhesive (e.g., polyisobutylene (“PIB”)) may be applied as the transfer adhesive layer 110 to mitigate a potential decrease in flexibility of the flexible backing 108. It can be seen that, in various embodiments, devices constructed in accordance with the present invention permit a degree of motion and flexure during treatment without disrupting the function of the assembly 100. The assembly 100 therefore exhibits low flexural rigidity in multiple directions, permitting conformability of the assembly 100 to a variety of membrane surface area configurations in a manner that is substantially independent of the chosen orientation of the assembly 100 during normal use. In various embodiments, the flexural rigidity of at least a portion of the flexible backing 108 is less than the flexural rigidity of at least a portion of the electrode layer 102.

In general, improvement in certain performance characteristics of certain embodiments of the present invention is realized in minimizing the “footprint” of the assembly 100 when the assembly 100 is applied to a membrane to deliver a composition. As applied herein, the term “footprint” refers to the portion or portions of the assembly 100 that contact a membrane surface area (e.g., a patient's skin) during operation of the assembly 100. In certain aspects, the surface area of an assembly including the donor electrode 104 and the donor reservoir 134 may be structured to be greater than the surface area of an assembly including the return electrode 106 and the return reservoir 134 to limit the effect of the return assembly on the overall footprint of the assembly 100. In addition, the length of the distance 152 that provides separation between the anode 104 and cathode 106 may also impact the footprint. Furthermore, the size of the electrodes 104, 106 relative to their respective reservoirs 134, 136 may also affect the footprint of the assembly 100. In certain aspects, the reservoirs 134, 136 should be at least substantially the same size as their respective electrodes 104, 106.

It can be appreciated that the inventors have also recognized that once the surface area of the electrode layer 102 is fixed, including configuration of the anode 104 and cathode 106 separation distance 152, the assembly 100 preferably should be sufficiently flexible and adherent for use on a membrane (e.g., a patient's skin). These objectives may depend on the peripheral area of the transfer adhesive layer 110 that surrounds the stiff electrode layer 102. In various embodiments, the width of the peripheral area of the transfer adhesive layer 110 adjacent to one or both of the anode 104 and cathode 106 may be provided as a minimum width 137 (as shown, for example, in FIG. 4). The minimum width 137 may be structured, in certain aspects, in the range of at least about 0.953 cm (0.375 inches). In turn, these objectives depend on the aggressiveness of the transfer adhesive layer 110 and the flexible backing 108, which is preferably flexible and compliant as a function of the strength (e.g., modulus of elasticity) and thickness of the flexible backing 108. Any sufficiently thin material may be flexible (such as ultra-thin PET, for example), but another problem arises in that the transfer adhesive layer 110 and the flexible backing 108 preferably are capable of removal from a membrane with minimum discomfort to a patient, for example. Consequently, a compliant (i.e., low strength) flexible backing 108 may be employed while maintaining adequate strength for treatments using the assembly 100.

The footprint area of the assembly 100 may be preferably in the range of about 3 cm² to 100 cm², more preferably in the range of about 5 cm² to 60 cm², and most preferably in the range of about 20 cm² to 30 cm². In addition, the total electrode 104, 106 area may be in the preferred range of about 2 cm² to 50 cm², or more preferably in the range of about 3 cm² to 30 cm², and most preferably in the range of about 4 cm² to 40 cm², respectively. In other aspects, the ratio of the area of each reservoir 134, 136 to its corresponding electrode 104, 106 may be, for example, in the range of about 1.0 to 1.5. In one operational example, the total contact area for the electrodes 104, 106 is about 6.3 cm² and the total reservoir 134, 136 contact area is about 7.5 cm². In other aspects, the flexible backing adhesive layer 110 for the printed electrode layer 102 may have a thickness in the range of, for example, about 0.0015 inches to about 0.005 inches. The flexible backing 108 may be comprised of a suitable material such as, for example, EVA, polyolefins, polyethylene (“PE”) (such as, for example, low-density polyethylene (“LDPE”), polyurethane (“PU”), and/or other similarly suitable materials.

According to other aspects of certain non-limiting embodiments according to the present invention, the ratio of total electrode surface area to total footprint area may be in the range about 0.1 to 0.7, or preferably about 0.24. In certain aspects, the ratio of donor electrode 104 surface area to return electrode 106 surface area may be in the range of about 0.1 to 5.0, or preferably about 1.7. In still other aspects, the ratio of donor reservoir 134 thickness to return reservoir 136 thickness may be in the range of about 0.1 to 2.0, or more preferably about 1.0.

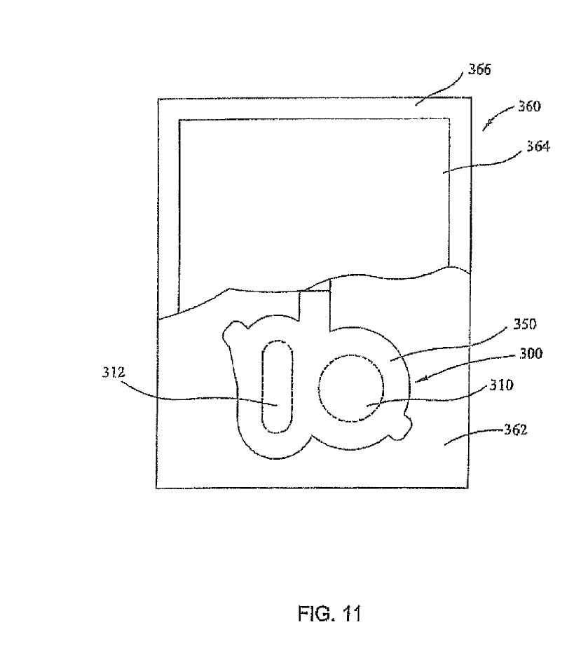

In various embodiments, the donor electrode reservoir 134, for example, may be loaded with an active ingredient from an electrode reservoir loading solution by placing an aliquot of the loading solution directly onto the hydrogel reservoir and permitting the loading solution to absorb and diffuse into the hydrogel over a period of time. FIG. 10 illustrates this method for loading of electrode reservoirs in which an aliquot of loading solution is placed on the hydrogel reservoir for absorption and diffusion into the reservoir. FIG. 10 is a schematic cross-sectional drawing of an anode electrode assembly 274 including an anode 280 and an anode trace 281 on a backing 288 and an anode reservoir 284 in contact with the anode 280. An aliquot of a loading solution 285, containing a composition to be loaded into the reservoir 284 is placed in contact with reservoir 284. Loading solution 285 is contacted with the reservoir 284 for a time period sufficient to permit a desired amount of the ingredients in loading solution 285 to absorb and diffuse into the gel reservoir 284. It can be appreciated that any suitable method or apparatus known to those in the art may be employed for loading the reservoir 284 with a composition.

In other embodiments of the present invention, at least one of the hydrogel reservoirs 134, 136 is positioned for electrical communication with at least a portion of at least one of the electrodes 104, 106. In various aspects, a surface area of at least one of the hydrogel reservoirs 134, 136 may be greater than or equal to a surface area of its corresponding electrode 104, 106. At least one of the hydrogel reservoirs 134, 136 may be loaded with a composition to provide a loaded hydrogel reservoir below an absorption saturation of the loaded hydrogel reservoir. In addition, at least one component of the assembly 100 in communication with, or in the vicinity of, the loaded hydrogel reservoir may have an aqueous absorption capacity less than an aqueous absorption capacity of the loaded hydrogel reservoir. In certain embodiments, a first kind of material comprising the unloaded hydrogel reservoir 134 in electrical communication with the anode electrode 104 is substantially identical to a second kind of material comprising the second unloaded hydrogel reservoir 136 in electrical communication with the cathode electrode 106.

In certain non-limiting embodiments of the present invention, a slit 202 may be formed in the flexible backing 108 in an area located between the anode 104 and the cathode 106 of the assembly 100. The slit 202 facilitates conformability of the assembly 100 to a membrane by dividing stress forces between the portion of the assembly including the anode and the portion of the assembly including the cathodes. In various embodiments, the electrode assembly 100 includes one or more non-adhesive tabs 206 and 208 that extend from the flexible backing 108 and to which no adhesive is applied. The non-adhesive tabs 206, 208 permit, for example, the ready separation of the release cover 138 from its attachment to the electrode assembly 100. The non-adhesive tabs 206, 208 also may facilitate removal of the assembly 100 from a membrane (e.g., a patient's skin) on which the assembly 100 is positioned for use.

As described above, at least a portion of at least one of the anode electrode trace 112 and the cathode electrode trace 114 may be covered with an insulating dielectric coating 118 at portions along the traces 112, 114. The insulating dielectric coating 118 may be structured not to extend to cover completely the portion of the traces 112, 114 located at the tab end portion 116 of the assembly 100. This permits electrical contact between the traces 112, 114 and the electrical contacts of an interconnect device such as the flexible circuit connector 250B of the connector assembly 250. In various embodiments, the dielectric coating 118 may cover at least a portion of at least one of the anode 104/reservoir 134 assembly and/or the cathode 106/reservoir 136 assembly. In addition, the dielectric coating 118 may cover substantially all or at least a portion of a periphery of at least one of the electrodes 104, 106 and/or the traces 112, 114.

In various embodiments of the present invention, a gap 212 may be provided between a portion of the layer of transfer adhesive 110 nearest to the tab end portion 116 and a portion of the tab stiffener 124 nearest to the layer of transfer adhesive 110 to facilitate removal or attachment of the assembly 100 from/to a component of an electrically assisted delivery device such as the connector assembly 250, for example. In certain example embodiments, the gap 212 is at least about 0.5 inches in width. The gap 212 provides a tactile sensation aid such as for manual insertion, for example, of the assembly 100 into the flexible circuit connector 250B of the connector assembly 250. The gap 212 may also provide relief from stress caused by relative movement between the assembly 100 and other components of a delivery device (e.g., the connector assembly 250) during adhesion and use of the assembly 100 on a membrane.

In addition, at least one tactile feedback notch 214 and one or more wings 216, 218 may be formed in or extend from the tab end 116 of the electrode assembly 100. The feedback notch 214 and/or the wings 216, 218 may be considered tactile sensation aids that facilitate insertion or removal of the tab end 116 into/from a component of an electrically assisted delivery device such as, for example, to establish an operative association with the flexible circuit connector 250B of the connector assembly 250. FIGS. 6B and 6C each show the layering of elements of the electrode assembly 100 as shown in FIG. 6. In FIGS. 6B and 6C, it can be seen that the thickness of layers is not to scale and adhesive layers are omitted for purposes of illustration. FIG. 6B shows a cross section of the anode electrode 104/reservoir 134 assembly and the cathode electrode 106/reservoir 136 assembly. The anode 104 and the cathode 106 are shown layered on the printed electrode layer 102. The anode reservoir 134 and the cathode reservoir 136 are shown layered on the anode 104 and the cathode 106, respectively. FIG. 6C is a cross-sectional view through the anode 104, the anode trace 112, and the anode reservoir 134. The anode 104, the anode trace 112 and a sensor trace 130 are layered upon the electrode layer 102. The anode reservoir 134 is shown in electrical communication with the anode 104. The tab stiffener 124, which may be composed of an acrylic material, for example, is shown attached to the tab end 116 of the assembly 100. In addition, the sensor trace 130 may be located at the tab end 116 of the electrode assembly 100.

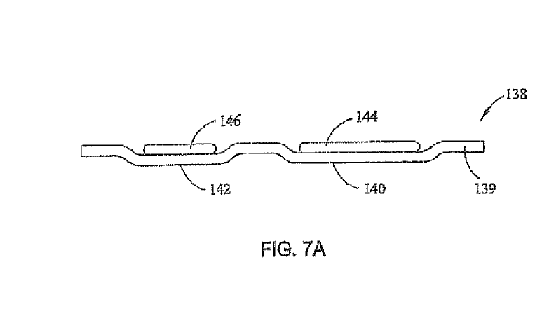

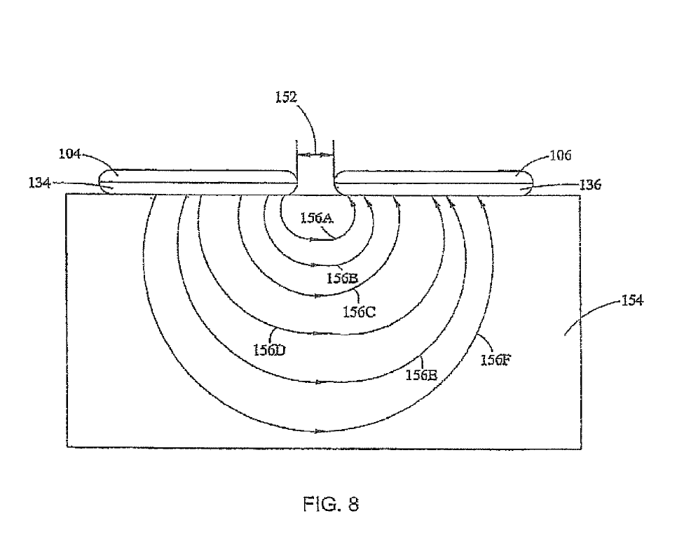

FIGS. 7 and 7A show schematically the release cover 138 structured for use with various embodiments of devices, electrode assemblies, and/or systems of the present invention. The release cover 138 includes a release cover backing 139, which includes an anode absorbent well 140 and a cathode absorbent well 142. In various exemplary aspects, a nonwoven anode absorbent pad 144 may be contained within the anode absorbent well 140 as the transfer absorbent, and a nonwoven cathode absorbent pad 146 may be contained within the cathode absorbent well 142 as the transfer absorbent. In use, the release cover 138 is attached to the electrode assembly 100 so that the anode absorbent pad 144 and the cathode absorbent pad 146 substantially cover the anode reservoir 134 and the cathode reservoir 136, respectively. The anode absorbent pad 144 and the cathode absorbent pad 146 may each be slightly larger than their corresponding anode reservoir 134 or cathode reservoir 136 to cover and protect the reservoirs 134, 136. The anode absorbent pad 144 and the cathode absorbent pad 146 may also be slightly smaller than the anode absorbent well 140 and the cathode absorbent well 142, respectively. In various embodiments, one or more indicia 220 (e.g., a “+” symbol as shown in FIG. 2) may be formed on at least a portion of the flexible backing 108 of the assembly 100 adjacent to the anode well 140 and/or the donor well 142. It can be appreciated that the indicia 220 may promote correct orientation and use of the assembly 100 during performance of an iontophoretic procedure, for example.

The anode absorbent pad 144 and the cathode absorbent pad 146 may be attached to the backing 139 of the release cover 138 by one or more ultrasonic spot welds such as welds 222, 224, 226, for example, as shown in FIG. 7. The welds 222, 224, 226 may be substantially uniformly distributed in areas of contact between the non-woven fabric pads 144, 146 and the wells 140, 142, respectively.

To facilitate removal of the release cover 138 from the electrode assembly 100, portions of the backing 139 in communication with the transfer adhesive 110 when the release cover 138 is attached to the electrode assembly 100 may be treated with a release coating, such as a silicone coating, for example.

Packaging of Iontophoretic Device

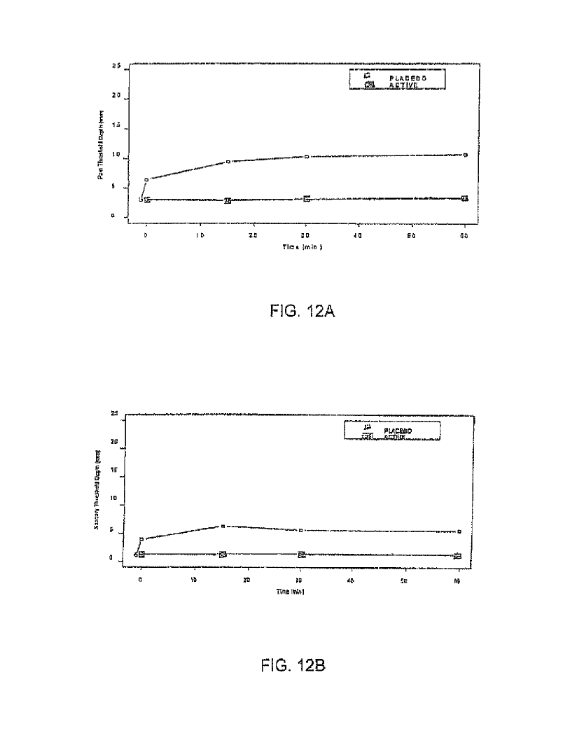

FIG. 11 is a breakaway schematic representation of an embodiment of an electrode assembly 300 constructed according to the invention within a hermetically sealed packaging 360. Packaged electrode assembly 300 is shown with release liner 350 in place and anode 310 and cathode 312 are shown in phantom for reference. Hermetically sealed packaging 360 is a container that is formed from a first sheet 362 and a second sheet 364, which are sealed along seam 366. Hermetically sealed packaging 360 can be of any suitable composition and configuration, so long as, when sealed, it substantially prevents permeation of any fluid or gas including, for example, permeation of oxygen into the packaging 360 and/or the loss of water from the packaging 360 after the electrode assembly 300 is sealed inside the hermetically sealed packaging 360.

In use, sheets 362 and 364 are sealed together to form a pouch after electrode assembly 300 is placed on one of sheets 362 and 364. Other techniques well-known to those skilled in the art of packaging may be used to form a hermetically sealed package with an inert atmosphere. In one embodiment, the moles of oxygen in the inert gas in the sealed pouch is limited by controlling the oxygen concentration in the inert gas and by minimizing the internal volume, or headspace, of the package, to be slightly less than an amount of sodium metabisulfite in the epinephrine-containing reservoir needed to react with all oxygen in the package. Electrode assembly 300 is then inserted between sheets 362 and 364, an inert gas such as nitrogen is introduced into the pouch to substantially purge air from the pouch, and the hermetically sealed packaging 360 is then sealed. The hermetically sealed packaging 360 may be sealed by adhesive by heat lamination, or by any method know to those skilled in the art of packaging drug delivery devices. It should be noted that sheets 362 and 364 may be formed from a single sheet of material that is folded onto itself, with one side of hermetically sealed packaging 360 being a fold in the combined sheet, rather than a seal. in other embodiments, the sheets 362, 364 may be formed from individual sheets that are laminated together, for example, to form a package. Other container configurations would be equally suited for storage of the electrode assembly, so long as the container is hermetically sealed.

Sheets 362 and 364, and, in general, hermetically sealed packaging 360 may be made form a variety of materials. In one embodiment, the materials used to form hermetically sealed packaging 360 have the following layered structure: 48 gauge PET/primer/15lb LDPE (low density polyethylene)/0.0254 mm (1.0 mil) aluminum foil/adhesive/48 gauge PET/10 lb LDPE chevron pouch 0.0508 mm (2 mil) peelable layer. Laminates of this type (foil, olefinic films, and binding adhesives) form strong and channel-free seals and are essentially pinhole-free, assuring essentially zero transfer of gases and water vapor for storage periods up to and exceeding 24 months. Other suitable barrier materials to limit transport of oxygen, nitrogen, and water vapor for periods of greater than 24 months are well-known to those of skill in the art, and include, without limitation, aluminum foil laminations, such as the Integra® brand products commercially available from Rexam Medical Packaging of Mundelein, Ill.

Protocols for Loading Reservoirs

It can be appreciated that any of the assemblies, devices, systems, and other apparatuses described herein may be, where structurally suitable, included within hermetically sealed packaging as described above.

In use, electrode reservoirs described herein can be loaded with an active ingredient from an electrode reservoir loading solution according to any method suitable for absorbing and diffusing ingredients into a hydrogel. Two possible methods for loading a hydrogel include, without limitation, placing the hydrogel in contact with an absorbent pad material, such as a nonwoven material, into which a loading solution containing the ingredients is absorbed. A second loading method includes the steps of placing an aliquot of the loading solution directly onto the hydrogel and permitting the loading solution to absorb and diffuse into the hydrogel over a period of time.

In applying the first method just mentioned to the electrode assembly 100, for example, the loading solution containing ingredients to be absorbed and diffused into the respective anode reservoir 134 and cathode reservoir 136 are first absorbed into the nonwoven anode absorbent pad 144 and nonwoven cathode absorbent pad 146, respectively. When a release cover thus loaded is connected to electrode assembly 100, the ingredients therein desorb and diffuse from the absorbent pads 144 and 146 and into the respective reservoirs. In this case, absorption and diffusion from the reservoir cover into the reservoirs has a transfer efficiency of about 95%, requiring that about a 5% excess of loading solution be absorbed into the absorbent pads. Despite this incomplete transfer, the benefits of this loading process, as compared to placing a droplet of loading solution onto the reservoirs and waiting between about 16 and 24 hours or so for the droplet to immobilize and absorb, can be significant because once the release cover is laminated onto the electrode assembly, the assembly can be moved immediately for further processing and placed in inventory. There is no requirement that the assembly is kept flat and immobile while awaiting completion of absorption and/or diffusion.

The transfer absorbents 144 and 146 are typically a nonwoven material. However, other absorbents may be used, including woven fabrics, such as gauze pads, and absorbent polymeric compositions such as rigid or semi-rigid open cell foams. In the particular embodiments described herein, as noted above, the efficiency of transfer of loading solution from the absorbent pads of the release cover to the reservoirs is about 95%. It will be appreciated by those skilled in the art that transfer efficiency will vary depending on the composition of the absorbent pads and the reservoirs as well as additional physical factors including, without limitation, the size, shape, and thickness of the reservoirs and absorbent pads and the degree of compression of the absorbent pads and reservoirs when the release cover is affixed to the electrode assembly. The transfer efficiency for any given release cover-electrode assembly combination can be readily determined empirically and, therefore, the amount of loading solution needed to fully load the reservoirs to their desired drug content can be readily determined to target specifications.

As discussed above, FIG. 10 illustrates the second method described above for loading of electrode reservoirs, wherein an aliquot of loading solution is placed on the hydrogel reservoir for absorption and diffusion into the reservoir. The transfer absorbents 144, 146 typically need not be included in the release cover for electrode assemblies having reservoirs loaded by this method.

In various embodiments, the electrode assembly 100 may be manufactured, in pertinent part, by the following steps. First, electrodes 104 and 106 and traces 112, 114 and 130 are printed onto a polymeric backing, such as treated ink-printable PET film, for example, or another suitably rigid material. The dielectric layer 118 may then be deposited onto the appropriate portions of traces 112 and 114 that are not intended to electrically contact the electrode reservoirs and contacts of an interconnect between the electrode assembly and a power supply/controller, for example. The polymeric backing onto which the electrodes are printed is then laminated to the flexible backing 108. The anode reservoir 134 and cathode reservoir 136 are then positioned onto the electrodes 104 and 106, respectively. In the assembly of the release cover 138, the transfer absorbents 144 and 146 are ultrasonically spot welded within wells 140 and 142 and are loaded with an appropriate loading solution for absorption and/or diffusion into the anode and/or cathode reservoirs 134 and 136. An excess of about 5% loading solution (over the amount needed to absorb and diffuse into the hydrogel) typically is added to the reservoir covers due to the about 95% transfer efficiency of the loading process, resulting in some of the loading solution remaining in the absorbent reservoir covers.

Once assembled and loaded with loading solution, the release cover is positioned on the electrode assembly 100 with the loaded transfer absorbents 144 and 146 in contact with anode and cathode reservoirs 134 and 136, respectively. Over a time period, typically at least about 24 hours, substantial portions (about 95%) of the loading solutions are absorbed and diffused into the hydrogel reservoirs. The completed assembly is then packaged in an inert gas environment and hermetically sealed.

Methods of Use

In one method of use, the release cover 138 is removed from the electrode assembly 100, and the electrode assembly 100 is placed on a patient's skin at a suitable location. After the electrode assembly 100 is placed on the skin, it is inserted into a suitable interconnect, such as a component of the connector assembly 250, for example. An electric potential is applied according to any profile and by any means for electrically assisted drug delivery known in the art or developed for delivery of a certain drug formulation. Examples of power supplies and controllers for electrically assisted drug delivery are well known in the art, such as those described in U.S. Pat. Nos. 6,018,680 and 5,857,994, among others. Ultimately, the optimal current density, drug concentration, and duration of the electric current and/or electric potential is determined and/or verified experimentally for any given electrode/electrode reservoir combination.

The electrodes described herein are standard Ag or Ag/Ag/Cl electrodes and can be prepared in any manner according to standard methods in a suitable ratio of Ag to AgCl (if initially present), thickness, and pattern, such that each electrode will support the Ag/AgCl electrochemistry for the desired duration of treatment. Typically, as is common in preparation of disposable iontophoresis electrodes, the electrodes and electrode traces are prepared by printing Ag/AgCl ink in a desired pattern on a stiff polymeric backing, for example print-treated 2 mm PET film, by standard lithographic methods, such as by rotogravure. Ag/AgCl ink is commercially available from E. I. du Pont de Nemours and Company, for example, as du Pont Product ID Number 5279. The dielectric also may be applied to the electrode traces by standard methods. As with the electrode, dielectric ink may be applied in a desired pattern over the electrodes and electrode traces by standard printing methods, for instance by rotogravure.

The pressure-sensitive adhesive (PSA) and transfer adhesives may be any pharmaceutically acceptable adhesive or adhesives suitable for the desired purpose. In the case of the pressure-sensitive adhesive, the adhesive may be any acceptable adhesive useful for affixing an electrode assembly to a patient's skin or other membrane. For example, the adhesive may be polyisobutylene (“PIB”) adhesive. The transfer adhesive, used to attach different layers of the electrode assembly to one another, also may be any pharmaceutically acceptable adhesive suitable for that purpose, such as PIB adhesive. For assembly of the electrodes described herein, the PSA typically is provided pre-coated on the backing material with a silicone-coated release liner attached thereto to facilitate cutting and handling of the material. Transfer adhesive typically is provided between two layers of silicone-coated release liner to facilitate precise cutting, handling, and alignment on the electrode assembly.

As noted above, the anode and cathode reservoirs described herein may comprise a hydrogel. The hydrogel typically is hydrophilic and may have varying degrees of cross-linking and water content, as is practicable. A hydrogel as described herein may be any pharmaceutically and cosmetically acceptable absorbent material into which a loading solution and ingredients therein can be absorbed, diffused, or otherwise incorporated and that is suitable for electrically assisted drug delivery. Suitable polymeric compositions useful in forming the hydrogel are known in the art and include, without limitation, polyvinylpyrrolidone (PVP), polyethyleneoxide, polyacylamide, polyacrylonitrile, and polyvinyl alcohols. In one non-limiting embodiment, the hydrogel may comprise from about 15% to about 17% by weight polyvinylpyrrolidone. The reservoirs may contain additional materials such as, without limitation: preservatives, such as Phenonip Antimicrobial, available commercially from Clariant Corporation of Mount Holly, N.C., antioxidants, such as sodium metabisulfite; chelating agents, such as ethylenediaminetetraacetic acid (“EDTA”); and humectants. A typical unloaded reservoir contains preservatives and salt. As used herein in reference to the water component of the electrode reservoirs, the water preferably is purified and preferably meets the standard for purified water in the USP XXIV.

As discussed above, the hydrogel has sufficient internal strength and cohesive structure to substantially hold its shape during processing, forming, and for its intended use and leaves essentially no residue when the electrode is removed after use. As such, the cohesive strength of the hydrogel and the adhesive strength between the hydrogel and the electrode are each greater than the adhesive strength of the bonding between the hydrogel and the membrane (for instance, skin) to which the electrode assembly is affixed in use. In one non-limiting embodiment, the hydrogel may have a thickness from about 0.89 mm (35 thousandths of an inch) to about 1.14 mm (45 thousandths of an inch).

The unloaded donor (anode) reservoir may include a salt, preferably a fully ionized salt, for instance a halide salt such as sodium chloride in a concentration of from about 0.001 wt. % to about 0.1 wt. %, preferably from about 0.01 wt. % to about 0.09 wt. %, and more preferably about 0.06 wt. %. The salt content is sufficient to prevent electrode corrosion during manufacture and shelf-storage of the electrode assembly. These amounts may vary for other salts in a substantially proportional manner depending on a number of factors, including the molecular weight and valence of the ionic constituents of each given salt in relation to the molecular weight and valence of sodium chloride. Other salts, such as organic salts, are useful in ameliorating the corrosive effects of certain drug salts. Typically the best salt for any ionic drug will contain an ion that is the same as the counter ion of the drug. For instance, acetates would be preferred when the drug is an acetate form. However, the primary aim is to prevent corrosion of the electrodes.

Lidocaine HCl and epinephrine bitartrate are used in the examples below to elicit a desired pharmacological response. If the counterion of lidocaine is not chloride, though chloride ions may be useful to prevent electrode corrosion, a corrosion-inhibiting amount of that other counterion may be present in the unloaded reservoir in addition to, or in lieu of the chloride ions to prevent corrosion of the electrode. If more than one counterion is present, such as in the case where more than one drug is loaded and each drug has a different counterion, it may be preferable to include sufficient amounts of both counterions in the reservoir to prevent electrode corrosion. It should be noted that in the examples provided below, the amount of epinephrine bitartrate loaded into the gel is not sufficient to cause corrosion.

The return (cathode) reservoir may be, for example, a hydrogel with the same or different polymeric structure and typically contains a salt such as sodium chloride, a preservative and, optionally, a humectant. Depending upon the ultimate manufacturing process, certain ingredients may be added during cross-linking of the hydrogel reservoir, while others may be loaded with the active ingredients. Nevertheless, it should be recognized that irrespective of the sequence of addition of ingredients, the salt must be present in the reservoir adhering to the electrode and substantially evenly distributed there through prior to the loading of the active ingredient(s) or other ingredient to limit the formation of concentration cells.

### Depth and Duration Study

A. Study Overview and Objectives

A study was conducted to assess the depth and duration of dermal anesthesia produced by an iontophoresis drug delivery system delivering a drug formulation including 10% lidocaine anesthetic and 0.1% epinephrine vasoconstrictor and producing an approximately 5 cm² region of local anesthesia on treated skin. The iontophoresis drug delivery system was constructed generally as shown in the attached FIGS. 2, 3, 4, 5, 5A-C, 6A-C, 7, and 7A, and as described in the foregoing text describing the device illustrated in those figures and as further described below in connection with this “Depth and Duration” study the iontophoretic drug delivery studies reported herein.

A primary objective of the study was to quantify the depth to which clinically meaningful anesthesia penetrates the skin immediately after treatment with the drug delivery system compared with a suitably designed placebo. A secondary objective of the study was to quantify the depth to which the sensation (such as pressure) is eliminated after treatment with the drug delivery system compared to placebo (which was an identical drug delivery device loaded with 0% lidocaine and 0.1% epinephrine), and to measure the depth of anesthetic effect over time from both pain and sensory perspectives.

In general, the study was a single center, prospective, placebo controlled, double blind clinical trial. Twenty evaluable subjects were enrolled at a single investigative site. Healthy Caucasian male and non-pregnant, non-nursing females, at least 18 years of age, able to complete all study procedures, including provision of informed consent, were enrolled. Subjects with any health condition that would impair their participation in the study, obvious marks, bruises or excessive hair at the treatment site or known sensitivity to amide anesthetics or epinephrine were excluded. Each subject received two treatments to the forearms in random order: an “active” treatment (i.e., iontophoretic delivery of the lidocaine/epinephrine formulation); and a “placebo” control treatment (i.e., iontophoretic delivery of a formulation lacking lidocaine but including epinephrine) in a paired comparison design. Both treatments were completed at a single visit. Skin temperature and thickness were assessed for each treatment. Baseline erythema (“EI”), cutaneous pain assessment (“CP”), sensory threshold depth (“SD”), and pain threshold depth (“PD”) were measured. The first treatment using the system (active or placebo) was applied and activated. At completion of the standard treatment cycle, the patch was removed and SD, PD, CP, EI, and skin temperature were measured in that order. All measurements were repeated at 15, 30, and 60 minutes after removal of the patch. Skin reactions were graded by Draize evaluation at the 60 minute time point. After the first treatment was completed all procedures were repeated on the contralateral forearm with the other (active or placebo) treatment. Subjects were seen approximately 24 hours later for repeat Draize evaluation of the skin.

A detailed discussion of the study and results follows.

B. Investigational Plan and Procedures

1. Study and Treatments

Subjects were enrolled and randomized to treatment order. Each subject received one treatment, active or placebo, to each forearm. The placebo control was an iontophoretic patch in which the lidocaine in the drug formulation in the drug reservoir was replaced with an equivalent amount of sodium chloride solution. The full electrical current delivery cycle was delivered to the placebo control iontophoretic patch.

Once the first treatment site was identified, skin temperature and thickness were assessed. Baseline EI and CP assessments were completed. A baseline SD and PD were measured. The first system treatment (active or placebo) was applied and activated according to written instructions. At completion of the treatment cycle, the patch was removed and SD and PD were assessed. CP, EI, and skin temperature were subsequently measured. All measurements were repeated at 15, 30 and 60 minutes after removal of the system patch. Skin reactions were graded by Draize evaluation at the 60 minute time point. All procedures were repeated on the contralateral forearm with the other (active or placebo) treatment. Subjects were seen approximately 24 hours later for repeat Draize evaluation of the skin.

a. Treatments Administered

Enrolled subjects received two treatments, one to each forearm as described further below. Every subject received one active and one placebo treatment, randomized by order and arm treated.

b. Identity of Study Treatments

Test articles were supplied as single-use pre-filled, integrated patches and reusable patch controllers, as specified in Table 1 below. It was determined to apply a charge density of between about 1.5 mA·min/cm² and about 4.2 mA·min/cm² for a time period of from about 5 to about 20 minutes.

TABLE 1
Identity of Study Treatments

| | |
|---|---|
| Drug | Lidocaine hydrochloride (100 mg in combination with 1.05 mg |
| Formulation: | epinephrine) in a crosslinked PVP hydrogel (containing glycerin, sodium |
| | metabisulfite, edentate disodium, citric acid, sodium chloride, and purified |
| | water). |
| Iontophoretic Delivery | |
| System: | |
| Integrated Patch: | Anode and cathode integrated into a single patch containing lidocaine and |
| | epinephrine, as generally described above with reference to FIGS. 2, 3, |
| | 4, 5, 5A-C, 6A-C, 7, and 7A. |
| Controller System: | Battery-operated device (100 mm × 60 mm × 25 mm, 130 g) delivered a |
| | preprogrammed current delivery profile while measuring the voltage |
| | across and the current through a patch during delivery, downloadable with |
| | last-use data capture. Delivered with a 3.4 mA-min/cm² charge density |
| Reference Therapy: | Placebo patch of same design as active patch, but including 0% lidocaine |
| | and 0.1% epinephrine in the drug reservoir. The lidocaine was replaced |
| | with NaCl. |

The iontophoretic drug delivery systems were composed of drug-filled patches connected to a direct current source (controller). The patch system used in this study was composed of an integrated patch where the anode and cathode resided in a single patch. The anode acted as the active drug delivery reservoir with the cathode as the return electrode. A peripheral adhesive surrounded the electrode and reservoirs and adhered the patches during the treatment. The peripheral adhesive helped the hydrogel reservoir maintain intimate contact with both the electrode and the skin during the applications. Active and placebo systems were identical with the exception of the anode on the patch, where lidocaine was replaced with sodium chloride solution in the placebo patches. Testing was performed on bilateral dorsal forearms.

c. Selection and Timing of the Dose for Each Subject

Each subject received two treatments, one active and one placebo. Order of treatment side (left or right) and type (active or placebo) were determined by a prospectively defined randomization schedule. The first and second treatments with the test system occurred in sequence rather than concurrently. The study schedule was designed so that no test measurements were performed during the active iontophoresis interval on the contralateral arm. The time between administration of the test system and meals or other activities was not specified in the protocol.

d. Treatment Compliance

Subjects were required to comply with their prescribed treatment regimen and to return to the study site for their follow-up visits. All subjects completed both study visits. Since study drug treatment was completed during a single clinic visit, treatment compliance was not an issue. The principal investigator and/or the sponsor had the option to discontinue a subject who was not compliant with the protocol, however, no such event occurred during the study.

2. Study Schedule

The study schedule and associated measurements are listed in Table 2.

TABLE 2
Study Schedule

| Visit Name | Interval | Procedures Performed —in order |
|---|---|---|
| Visit 1 | Intake | Informed Consent |
| | | Inclusion/Exclusion Evaluation |
| | | Medical History Interview |
| | Baseline measurements for first arm | Randomization |
| | | Skin temperature clearance |
| | | Skin thickness measurement (× 2) |
| | | Erythema Index (EI) |
| | | Cutaneous Pain assessment (CP) |
| | | Sensory Depth threshold (SD) |
| | | Pain Depth threshold (PD) |
| | Iontophoresis system treatment to first arm | According to written instructions |
| | Testing: | SD |
| | T = 0 (immediately post patch removal) | PD |
| | T = 15 minutes | CP |
| | T = 30 minutes | EI |
| | | Skin temperature |
| | T = 60 minutes | SD |
| | | PD |
| | | CP |
| | | EI |
| | | Skin temperature |
| | | Draize skin evaluation |
| | Baseline measurements for second arm | Skin temperature clearance |
| | | Skin thickness measurement (× 2) |
| | | EI |
| | | CP |
| | | SD |
| | | PD |
| | Iontophoresis system treatment to second | According to written instructions |
| | arm | |
| | Testing: | SD |
| | T = 0 (immediately post patch removal) | PD |
| | T = 15 minutes | CP |
| | T = 30 minutes | EI |
| | | Skin temperature |
| | T = 60 minutes | SD |
| | | PD |
| | | CP |
| | | EI |
| | | Skin temperature |
| | | Draize skin evaluation |
| Visit 2 | Approx. 24 hours (±4 hours) after treatment | Draize skin evaluation |
| | #1 | |

3. Description of Variables

In addition to general data on the various enrollees, the following variables were captured according to the schedule defined in Table 2 above.

a. Efficacy Measurements

SD and PD measurements were taken according to the procedure described below. They were recorded immediately after removal of the patch after a treatment and at 15, 30 and 60 minutes thereafter.

Depth measurements were obtained utilizing a specifically designed fixture, which was hidden from the subject's view by a drape. An 18 gauge needle was attached to a mount on the fixture. A 3 cm diameter footplate rested on the skin in the test area with a constant pressure of 200 grams, limiting dimpling of the skin during needle insertion. The needle was advanced perpendicular to the skin at a stable rate of 0.2 mm/sec with the aid of a computer controlled step motor. The subject was asked to press a button to indicate initial perception of pressure (SD) and of pain (PD). A micrometer with a resolution of 0.01 mm was mounted on the fixture to measure the depth of needle penetration whenever the button was triggered. All measurements from the fixture were recorded and stored in the attached computer system for later download as supporting source documentation. A new needle was used for each challenge.

Pain threshold depth (PD) is the parameter that was measured to support the primary objective for the study. After SD was recorded as described below, the needle continued to advance. The subject was asked to press a button on the apparatus again, at the onset of a pain sensation, which triggered the needle to stop advancing. The maximum depth of needle penetration was recorded digitally for later retrieval, and the needle was retracted automatically. Sensory threshold depth (SD) was measured with the same instrument. Subjects were asked to press a button attached to the apparatus when they first felt any sensation of pressure or touch at the test region, and the depth measurement was recorded digitally.

All depth measurements were performed in an identical manner at baseline, immediately after removal of the patch after treatment, and 15, 30 and 60 minutes thereafter. The primary objective was assessed by measuring and recording PD and SD immediately after patch removal (T=0). Subsequent measures supported the study's secondary objective, which was to measure the durability of any observed effect.

The following data were collected to document control of dermal conditions over the course of the study. All assessments were performed on the same schedule as the depth measures described above.

Cutaneous Pain Assessments—Cutaneous pain assessment (CP) was obtained by pressing a #18 von Frey hair to the test region for 3 seconds with a force of 0.18 mN, which corresponds to the bending force of the filament. In normal, non-anesthetized skin this stimulus causes a pricking pain sensation. The intensity of this stimulus was recorded by the subject on a 10 cm Visual Analog Scale (VAS).

Erythema Index—The Erythema index (EI) was measured with a Dermaspectrometer device (Cortex Technology A/S, Denmark), in standardized arbitrary units. The measurement was non-invasive.

Skin Temperature—Temperature of the skin at the test region was measured by a digital direct-contact thermocouple thermometer. The target range for the temperature measure was 29-34° C. (84.2-93.2° F.). Subjects who presented at baseline outside of this range were permitted time to normalize their skin to ambient temperature. If they failed to meet this criterion after a reasonable time point, at the discretion of the investigator, they were to be withdrawn and discharged. No such withdrawals occurred.

Skin Thickness—Duplicate measures of skin thickness at the test region were performed at baseline with a standard pinch caliper.

b. Safety Measurements

The skin site exposed to iontophoretic treatment was evaluated for erythema and edema using the Draize scale at 60 minutes post-treatment on the test day, and at the follow-up visit. Subjects who were evaluated with a Draize score of 4 would have been considered to receive a “burn”. No “burn” events were recorded during the study.

An “adverse event” was defined as any unintended, unfavorable clinical sign, symptom, medical complaint or clinically relevant change in laboratory test value, whether or not considered to be test article related. Potential adverse events were assessed at visit 1 and visit 2 according to the protocol. No adverse events were recorded in this clinical trial.

4. Statistical Methods and Determination of Sample Size

a. Statistical and Analytical Plan

The hypotheses used to evaluate the study objectives were expressed as:
H₀:μ_{PD}^{(N)}=μ_{PD}^{(P)}
Hₐ:μ_{PD}^{(N)}>μ_{PD}^{(P)}
and
H₀:μ_{SD}^{(N)}=μ_{SD}^{(P)}
Hₐ:μ_{SD}^{(N)>μ}_{SD}^{(P)}
where N is the active system, P is the placebo, μ_{PD} is the mean Pain Depth Threshold measurement and μ_{SD} is the mean Sensory Depth Threshold measurement. In addition a time by treatment interaction effect was assessed. Data was included in analysis supporting the primary objective only when both members of a treatment pair were evaluated.

Pain threshold depth (PD) was the parameter measured to support the primary objective for the study. Sensory threshold depth (SD) was measured to support a secondary objective for the study. Analysis of Variance was used to detect differences in depth of pain and touch perception between active and placebo. SD and PD were measured at 5 time points: baseline, immediately after removal of the patch system after treatment, 15 minutes, 30 minutes and 60 minutes thereafter. Change profile over time was charted for active and placebo treatments within each subject and across all subjects.

Average skin thickness at the test regions was analyzed in relation to depth measurements to identify any potential interaction effect. Visual Analog Scale results of COP were assessed within each subject over time. Any recognized interaction effects were recorded and investigated. Relationship to depth measures was to be specifically evaluated. EI results were assessed within each subject over time. Any recognized interaction effects were recorded and investigated. Relationship to depth measures was to be specifically evaluated. Dermal effects of active and placebo treatment were evaluated overtime, and identification of trends was attempted.

b. Determination of Sample Size

It was determined that based on an estimated sigma of 0.70 mm and α=0.05, a sample size of n=20 subjects would provide at least 90% power to detect a difference of 0.1 mm between placebo and active treatment with the iontophoresis system.

C. Study Subjects

1. Disposition of Subjects

Twenty evaluable subjects were assessed in the study. Twenty-one subjects were enrolled. One subject was withdrawn when the second treatment was manually discontinued due to operator error. Data from this subject was not included in the efficacy analysis. The remaining 20 subjects successfully completed all study procedures and assessments, including the requirements for follow-up. Treatments followed the predefined randomization schedule summarized in Table 3.

TABLE 3
Distribution of Randomization

| Factor | Active | Placebo |
|---|---|---|
| n = 21 | # (%) | # (%) |
| Side: Right | 10 (47.6%) | 11 (52.4%) |
| Left | 11 (52.4%) | 10 (47.6%) |
| Order: First | 11 (52.4%) | 10 (47.6%) |

2. Protocol Deviations

There were no obvious, consistent, or systematic deviations in protocol compliance that could serve as a source of bias. T=0 was defined as the point in time where the iontophoresis patch was removed, and all study assessments were conducted based on that time reference. For logistical purposes, a convention for assessment timing was defined prospectively as plus or minus 5 minutes for measurements at T=15, T=30, and T=60. Several deviations were outside the reference window, but no measurements were excluded from the analysis based on the timing deviation.

D. Efficacy and Safety Evaluations

1. Data Sets Analyzed

Evaluability of subjects was determined before the blind was broken. The study protocol stipulated that data were to be included in efficacy analyses only when both members of a treatment pair were available. Twenty of 21 subjects were included in the efficacy analysis. Subject 20 was not included in analysis since the second treatment was incomplete due to operator error.

2. Demographic Data and Baseline Evaluations

The demographic characteristics of the study population are summarized in Table 4.

TABLE 4
Demographic Characteristics

| Variable | Number (%), n = 21 |
|---|---|
| Gender: | |
| Male | 5 (24%) |
| Female | 16 (76%) |
| Age (years): | |
| Mean ± SD | 43.0 ± 10.8 |
| Range | 20-60 |
| Race: | |
| Caucasian | 21 (100%) |
| Height (inches):* | |
| Mean ± SD | 66.3 ± 3.4 |
| Range | 62-75 |
| Weight (pounds):* | |
| Mean ± SD | 185.1 ± 42.5 |
| Range | 105-250 |
| Body Mass Index:* | |
| Mean ± SD | 29.6 ± 6.7 |
| Range | 17.5-39.2 |

*Data collected on enrollment in study, which preceded the study trials by up to two years.

At baseline, all of the subjects' skin sites chosen for testing were clear and intact. None of the subjects had scars, moles, excessive pigmentation, excessive hair, bruising, abrasions, or wounds in the treatment areas.

3. Concomitant Medications and Conditions

The study was conducted on healthy volunteers. One subject reported concomitant medication use, while two others reported a concurrent medical condition. No condition or concomitant medication was considered to impact study assessments.

4. Primary Efficacy Endpoints

a. Pain Depth Threshold at T=0

A fixture was used to administer the painful challenge to the test area as described above. Immediately after removal of the iontophoretic electrode assembly, the fixture was applied, subjects were asked to indicate the point at which they first experienced a painful sensation (PD), and the fixture recorded the depth to which an 18 gauge needle penetrated the skin at that point. PD after active iontophoresis treatment was compared to PD after placebo treatment. Results are summarized in Table 5. The listed “difference” value in this and further tables below is the difference of the listed means (active−placebo), in millimeters.

TABLE 5
PD Results at T = 0

| | Active | Placebo |
|---|---|---|
| N | 20 | 20 |
| Mean (mm) | 6.37 | 3.09 |
| Max (mm) | 12.08 | 6.38 |
| Min (mm) | 2.35 | 1.31 |
| SD | 2.72 | 1.42 |

| | |
|---|---|
| Difference (mm) | 3.28 |
| SE of the difference | 0.58 |
| p value of difference | <0.0001 (one sided) |

Analysis of variance (ANOVA) was performed with subject, order, side, skinfold thickness and treatment as factors. Order of treatment (first or second) and treatment (active or placebo) both had significant effect on the response. Anesthesia was significantly deeper following active treatment (Mean PD=6.37 mm) as compared to placebo treatment (Mean PD=3.09 mm). Based on confidence intervals, it can be stated with 95% certainty that the depth of anesthesia penetration at T=0 is at least 2.26 mm deeper for active treatment than for placebo at T=0.

5. Secondary Efficacy Endpoints

a. Pain Depth Threshold at Baseline, T=15, T=30, and T=60

Along with the primary objective (PD at T=0), PD was measured before treatment (baseline), and 15, 30, and 60 minutes after removal of the electrode assembly. Results are summarized in Table 6. The “95% limit” in this and further tables below is the lower bound of the 95% confidence limit for the difference, in millimeters.

TABLE 6
PD Results to Support the Secondary Objective

| | Baseline | | T = 15 | | T = 30 | | T = 60 | |
|---|---|---|---|---|---|---|---|---|
| | Active | Placebo | Active | Placebo | Active | Placebo | Active | Placebo |
| N | 20 | 20 | 20 | 20 | 20 | 20 | 20 | 20 |
| Mean (mm) | 2.91 | 3.16 | 9.38 | 2.87 | 10.33 | 3.24 | 10.68 | 3.35 |
| Max (mm) | 5.08 | 6.49 | 25.00 | 4.74 | 25.00 | 6.16 | 24.30 | 11.86 |
| Min (mm) | 1.01 | 1.52 | 3.30 | 1.19 | 4.93 | 0.98 | 4.22 | 0.72 |
| SD | 1.13 | 1.22 | 6.31 | 1.07 | 5.18 | 1.36 | 6.35 | 2.35 |

| | | | | |
|---|---|---|---|---|
| Difference | −0.25 | 6.51 | 7.09 | 7.33 |
| 95% limit | | 4.03 | 4.61 | 4.85 |

In two cases, in different subjects, the test fixture reached its maximum insertion depth (25 mm) before the subject indicated any pain perception. One case occurred at T=15, and the other at T=30, both in sites with active iontophoresis treatment. In these cases the maximum recordable score (25 mm) was used in the calculations.

b. Sensory Threshold Depth at Baseline, T=0, T=15, T=30, T=60

The fixture used to record PD also was used to assess the depth to which sensation was removed entirely. This measurement was collected immediately preceding, and in the same procedure, as the PD challenge. During testing, subjects were asked to indicate the point at which they first experienced any sensation (SD), usually described as pressure, and the fixture recorded the depth to which an 18 gauge needle penetrated the skin at that point. SD after active iontophoresis treatment was also compared to SD after placebo treatment. Results are summarized in Table 7.

TABLE 7
SD Results to Support the Secondary Objective

| | Baseline | | T = 0 | | T = 15 | | T = 30 | | T = 60 | |
|---|---|---|---|---|---|---|---|---|---|---|
| | Active | Placebo | Active | Placebo | Active | Placebo | Active | Placebo | Active | Placebo |
| N | 20 | 20 | 20 | 20 | 20 | 20 | 20 | 20 | 20 | 20 |
| Mean (mm) | 0.98 | 1.21 | 3.90 | 1.35 | 6.27 | 1.34 | 5.58 | 1.39 | 5.58 | 1.29 |
| Max (mm) | 2.77 | 3.79 | 6.37 | 4.75 | 24.30 | 3.89 | 14.68 | 3.91 | 17.53 | 4.56 |
| Min (mm) | 0.04 | 0.13 | 1.01 | 0.19 | 1.64 | 0.15 | 0.27 | 0.16 | 1.14 | 0.12 |
| SD | 0.79 | 1.02 | 1.60 | 1.29 | 5.43 | 0.89 | 3.09 | 0.83 | 3.45 | 1.09 |

| | | | | | |
|---|---|---|---|---|---|
| Difference | −0.23 | 2.55 | 4.93 | 4.19 | 4.29 |
| 95% limit | | 0.88 | 3.27 | 2.53 | 2.62 |

Analysis of Variance (ANOVA) was performed with subject, order, side, skinfold thickness, time and treatment as factors. Treatment (active or placebo) was the only factor identified as having a significant effect. The average difference between the means at T=0 was statistically significant at 2.55 mm (p<0.0001), increased by T=15, and remained essentially stable throughout the remainder of the measurement interval.

c. Duration of Effect

PD and SD measurements were collected overtime after treatment to assess the durability of the anesthetic response. As illustrated in FIG. 12A, PD for placebo treatment remained essentially unchanged from baseline through T=60. Although the primary objective assessed PD at T=0, the data indicate that the active iontophoresis treatment mean PD increased throughout the measurement interval, demonstrating stable improved anesthesia as compared to placebo. SD results were consistent with those observed for PD, and are illustrated in FIG. 12B.

6. Supportive Efficacy Variables

a. Cutaneous Pain Assessment at Baseline, T=0, T=15, T=30, T=60

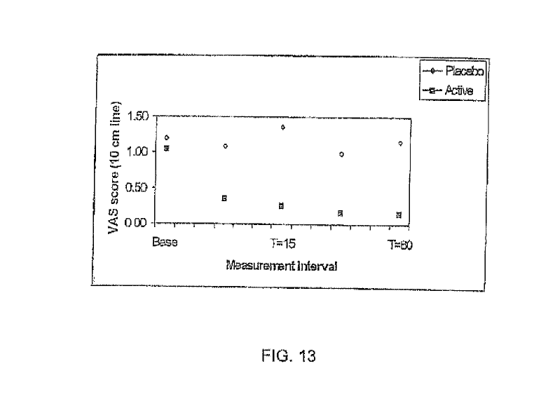

Cutaneous pain (CP) was measured at all treatment intervals to assess if there was a relationship between subject reports of pain sensitivity and measured PD. A von Frey hair was pressed against the intact skin in the test region at a pressure known to result in a pricking painful sensation. Subjects were asked to rate the pain they experienced on a 10 cm visual analog scale (VAS), where 0 indicated “no pain” and 10 was “severe pain”, CP results showed a negative correlation to SD (−0.42, p-value=0.045) at baseline; that is, as baseline perception of painful stimulus at the surface of the skin becomes more severe, the depth at which perception of pressure is first noted is less. No other correlation was observed for either SD or PD at any time point. FIG. 13 illustrates CP observations over time.

b. Erythema Index at Baseline, T=0, T=15, T=30 and T=60

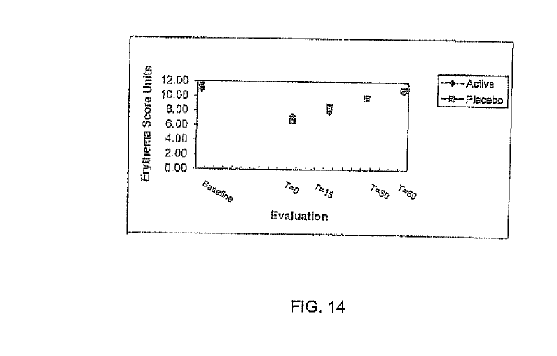

Erythema was measured at all 5 assessment periods during testing to assess the potential relationship between vascularity and sensitivity to dermal stimuli. Values were collected with a Dermaspectrometer Erythema Meter, and recorded in arbitrary erythema units (EI). An ANOVA was performed to assess significance of subject, order, side, skinfold thickness, time and treatment to the observations. Subject and time were identified as factors influencing the result. Treatment identity was specifically not a factor. FIG. 14 summarizes the observations overtime. No correlation was identified between EI and either PD or SD.

7. Efficacy Conclusions

The primary objective of the study was to quantify the depth to which clinically meaningful anesthesia penetrates the skin after active iontophoresis treatment, and compare that depth to placebo. Pain threshold depth (PD) was measured immediately after treatment and found to average 6.37 mm, an increase of 3.28 mm from placebo treatment at the same time point. This difference is statistically significant (p<0.0001). The depth to which all sensation was eliminated (SD) was also quantified (average 3.90 mm), and found to be increased 2.55 mm in comparison to placebo at T=0, another statistically significant difference (p<0.0001).

Neither cutaneous perception of pain (CP), vascularity in the region (EI), side of treatment, nor skin thickness had a significant effect on the measurement. Further, the depth of anesthesia penetration was assessed at intervals for one hour after conclusion of treatment. Pain threshold depth increased throughout the measurement period, reaching an average depth of 10.68 mm (a 7.33 mm increase from placebo), and demonstrating substantial duration of the effect. Sensory threshold depth performed consistently with PD, increasing to a maximum of 6.27 mm, a 4.93 mm increase from placebo.

8. Safety Results—Dermal Effects

The Draize scoring system used during the study is displayed in Table 8 below. Increasing scores on a 0-4 scale indicate increasing levels of erythema and edema.

TABLE 8
Draize Score System

| Erythema Formation | | Edema Formation | |
|---|---|---|---|
| Description | Score | Description | Score |
| No Erythema | 0 | No Edema | 0 |
| Very slight erythema | 1 | Very slight edema | 1 |
| (barely perceptible) | | (barely perceptible) | |
| Well defined erythema | 2 | Well defined edema | 2 |
| | | (edges of area well | |
| | | defined raising) | |
| Moderate to severe erythema | 3 | Moderate edema | 3 |
| | | (raised approx. 1 mm) | |
| Severe erythema | 4 | Severe edema | 4 |
| (beet redness to slight eschar | | (raised more than 1 mm | |
| formation) | | and beyond exposure | |
| | | area) | |

Listings of dermal effects (Draize scores of erythema and edema) at T=60 minutes and T=24 hours post treatment are reported in Tables 9A and 9B.

TABLE 9A
Draize Score Summary (Anode)

| | | | |
|---|---|---|---|
| | Draize | Active (n = 20) | Placebo (n = 20) |
| Time | Score | N (%) | N (%) |

Anode Erythema

| | | | | | |
|---|---|---|---|---|---|
| T = 60 | 0 | 19 | (95%) | 17 | (85%) |
| | 1 | 1 | (5%) | 3 | (15%) |
| | 2 | 0 | | 0 | |
| | 3 | 0 | | 0 | |
| | 4 | 0 | | 0 | |
| T = 24 | 0 | 20 | (100%) | 20 | (100%) |
| hours | 1 | 0 | | 0 | |
| | 2 | 0 | | 0 | |
| | 3 | 0 | | 0 | |
| | 4 | 0 | | 0 | |

Anode Edema

| | | | | | |
|---|---|---|---|---|---|
| T = 60 | 0 | 20 | (100%) | 20 | (100%) |
| | 1 | 0 | | 0 | |
| | 2 | 0 | | 0 | |
| | 3 | 0 | | 0 | |
| | 4 | 0 | | 0 | |
| T = 24 | 0 | 18 | (90%) | 20 | |
| hours | 1 | 2 | (20%) | 0 | |
| | 2 | 0 | | 0 | |
| | 3 | 0 | | 0 | |
| | 4 | 0 | | 0 | |

TABLE 9B
Draize Score Summary (Cathode)

| | | | |
|---|---|---|---|
| | Draize | Active (n = 20) | Placebo (n = 20) |
| Time | Score | n (%) | N (%) |

Cathode Erythema

| | | | | | |
|---|---|---|---|---|---|
| T = 60 | 0 | 12 | (60%) | 12 | (60%) |
| | 1 | 8 | (40%) | 8 | (40%) |
| | 2 | 0 | | 0 | |
| | 3 | 0 | | 0 | |
| | 4 | 0 | | 0 | |
| T = 24 | 0 | 20 | (100%) | 20 | (100%) |
| hours | 1 | 0 | | 0 | |
| | 2 | 0 | | 0 | |
| | 3 | 0 | | 0 | |
| | 4 | 0 | | 0 | |

Cathode Edema

| | | | | | |
|---|---|---|---|---|---|
| T = 60 | 0 | 20 | (100%) | 20 | (100%) |
| | 1 | 0 | | 0 | |
| | 2 | 0 | | 0 | |
| | 3 | 0 | | 0 | |
| | 4 | 0 | | 0 | |
| T = 24 | 0 | 20 | (100%) | 20 | |
| hours | 1 | 0 | | 0 | |
| | 2 | 0 | | 0 | |
| | 3 | 0 | | 0 | |
| | 4 | 0 | | 0 | |

Skin effects were limited to a small number of Draize 1 scores at T=60. Observed effects had resolved by the 24-hour evaluation in all cases. There was no statistical difference between the distribution of scores for active and placebo treatments; in most cases they were identical,

E. Overall Study Conclusions

The study confirmed that the iontophoresis electrode assembly produced clinically acceptable depth and duration of anesthesia on the treated skin site. A purpose of the study was to develop quantitative insight on the performance of the tested iontophoresis system and drug formulation. The difference between mean anesthesia penetration depths for active and placebo iontophoresis treatments (6.37 mm vs. 3.09 mm) at T=0 is considered clinically meaningful. Since the duration of the anesthesia effect exceeded the 60-minute post-treatment measurement interval with the active treatment, the durability of the anesthesia effect with the active treatment also was significant. No safety issues were identified during the study.

### Stability

As briefly mentioned above, “stability” may refer to a variety of qualities of the reservoir-electrode. Drug or pharmaceutical stability is one parameter. For instance, epinephrine typically is very unstable. Therefore, an iontophoretic electrode assembly might be considered stable for the time period that useful quantities of epinephrine remain available for delivery. Similarly, if lidocaine is considered, the electrode assembly remains stable for the time period that useful quantities of lidocaine remain available for delivery.

Physical stability also may be considered. Hydrogel strength (for example, apparent compressive modulus, as shown in the Examples) and probe tack are examples of the parameters considered for physical stability. In the case of electrical and/or electrochemical stability, retention of useful electrochemical capacity (specific capacity; mA·min/cm²) and trace conductance may be measured. As discussed above, though the FDA requires specific statistical tests and limits to permit an iontophoretic device to be marketed as stable, those standards are examples of what is considered to be a stable parameter, stability referring to retention of a parameter within desired boundaries to remain functional. This typically is a range of given properties, for example as shown in the Examples below.

Described with specificity herein is an embodiment of an iontophoretic system for delivery of the topical anesthetic lidocaine with the vasoconstrictor epinephrine, more specifically lidocaine HCl and epinephrine bitartrate as shown in the Examples. The particular amounts of epinephrine and lidocaine shown in the Examples are selected to produce effective local anesthesia. According to one non-limiting embodiment, the reservoir may comprise lidocaine HCl and epinephrine bitartrate in about a 50-1000:1 mass ratio. In another non-limiting embodiment the reservoir may comprise lidocaine HCl and epinephrine bitartrate in about a 70-125:1 mass ratio. Variations in the relative concentration and/or mass of lidocaine and/or epinephrine, as well as variations in reservoir volume, reservoir composition, reservoir skin contact surface area, electrode size and composition, and electrical current profile, among other parameters, could result in changes in the optimal concentrations of lidocaine and/or epinephrine in the gel reservoir. A person of skill in the art would be able to adjust the relative amounts of ingredients to achieve the same results in a system in which any physical, electrical, or chemical parameter differs from those disclosed herein.

For most, if not all applications, epinephrine stability should not be dependent upon epinephrine concentration within a range that can be extrapolated from the data provided herein. An example of a useful range of epinephrine is, therefore, from about 0.01 mg/ml to about 2.0 mg/ml.

Although lidocaine is a common topical anesthetic, other useful topical (surface and/or infiltration) anesthetics may be used in the described system. These anesthetics include, without limitation, salts of: amide type anesthetics such as bupivacaine, butanilicaine, carticaine, cinchocaine/dibucaine, clibucaine, ethyl parapiperidino acetylaminobenzoate, etidocaine, lidocaine, mepivicaine, oxethazaine, prilocaine, ropivicaine, tolycaine, trimecaine, and vadocaine; ester type anesthetics, including esters of benzoic acid such as amylocaine, cocaine, and propanocaine, esters of metaaminobenzoic acid such as clormecaine and proxymetacaine, esters of paraaminobenzoic acid (PABA) such as a methocaine (tetracaine), benzocaine, butacaine, butoxycaine, butyl aminobenzoate, chloroprocaine, oxybuprocaine, parethoxycaine, procaine, propoxycaine and tricaine; and miscellaneous anesthetics, such as, bucricaine, dimethisoquin, diperodon, dyclocaine, ethyl chloride, ketocaine, myrtecaine, octacaine, pramoxine, and propipocaine.

Of the topical anesthetics, salts of bupivacaine, butacaine, chloroprocaine, cinchocaine, etidocaine, mepivacaine, prilocaine, procaine, ropivacaine and tetracaine (amethocaine) might be considered by some to be more clinically relevant than other anesthetics listed above, though not necessarily more effective. Certain other features of each of the compounds listed above may make any particular compound more or less suited to iontophoretic delivery as described herein. For example, use of cocaine may be contra-indicated because of its cardiovascular side effects. Bupivacaine, butacaine, chloroprocaine, cinchocaine, etidocaine, mepivacaine, prilocaine, procaine, ropivacaine and tetracaine (amethocaine) may be preferred as a substitute for lidocaine because these anesthetics all have similar pKs of about 8 or greater than 8, meaning they will ionize under the same conditions as lidocaine. Iontophoresis in vitro across human skin has shown that bupivacaine and mepivacaine show a similar cumulative delivery as lidocaine, while etidocaine, prilocaine and procaine have shown slightly greater delivery. Chloroprocaine, procaine and prilocaine have similar relatively short duration effects (less than 2 hr) whereas bupivicaine, etidocaine, and mepivacaine have effects lasting 3-4 hr. These times are approximately doubled when epinephrine is used in conjunction with these anesthetics. The duration of the action of the local anesthetic is dependent upon the time for which it is in contact with the sensory nerves. This duration of effect will depend on the physiochemical and pharmacokinetic properties of the drug. Hence, any procedure that can prolong contact between the therapeutic agent and the nerve, such as co-delivery of a vasoconstrictor with the anesthetic, will extend the duration of action.

Another factor that should be considered is that ester-based anesthetics based on PABA are associated with a greater risk of provoking an allergic reaction because these esters are metabolized by plasma cholinesterase to yield PABA, a known allergen. For this reason, amide anesthetics might be preferred, and molecules such as chloroprocaine and procaine would not be viewed as first-line replacements for lidocaine. Because bupivacaine, etidocaine, mepivacaine, ropivicaine and prilocaine are amide anesthetics with similar physiochemical properties and clinical effects as lidocaine, they may be preferred by some as substitutes for lidocaine. A secondary issue with prilocaine is that although it is generally considered to be the safest of the amide anesthetics, one of its metabolites (o-toluidine) has been associated with increased risk of methemoglobinemia and cyanosis as compared to the other amide anesthetics.

Each of the anesthetics listed above have varying degrees of vasoconstrictor activity. Therefore, optimal concentrations of the anesthetic and the vasoconstrictor will vary depending on the selected local analgesic. However, for each local anesthetic, optimal effective concentration ranges can be readily determined empirically by functional testing. As used herein, the terms “anesthetic” and “anesthesia” refer to a loss of sensation, and are synonymous with “analgesics” and “analgesia” in that a patient's state of consciousness is not considered when referring to local effects of use of the described iontophoretic device, even though some of the drugs mentioned herein may be better classified as “analgesics” or “anesthetics” in their systemic use. Sodium metabisulfite may be added to the donor reservoir to scavenge oxygen. The amount of sodium metabisulfite added is not substantially in excess of the amount needed to scavenge all oxygen from the packaged reservoir for a given time period to minimize the formation of the adduct epinephrine sulfonic acid, and/or other decomposition products. For example, the donor hydrogel may contain less than about 110%, for example about 101%, of the amount of sodium metabisulfite equal to a minimal amount of sodium metabisulfite needed to scavenge substantially all oxygen in the packaged donor hydrogel. The amount of sodium metabisulfite needed to scavenge oxygen in the packaged donor hydrogel for any given amount of time can be calculated from the amount of oxygen present within the package in which the donor hydrogel is hermetically sealed. Alternately, the optimal amount of sodium metabisulfite can be titrated by determining the amount of sodium metabisulfite at which production of the oxidation products of epinephrine, due to its reaction with oxygen, such as adrenolone or adrenochrome, and epinephrine sulfonic acid, essentially stops.

Patch Fabrication Platform I—Pre-Loaded Integrated Patch

FIGS. 2 through 11 illustrate various aspects of an integrated electrode assembly 100 structured for use with an electrically assisted delivery device, for example, for delivery of a composition through a membrane. Patch Fabrication Platform I corresponds to an integrated patch as shown in, and as described herein in connection with, FIGS. 2 through 11, except for FIG. 10. The integrated patch of Patch Fabrication Platform I is pre-loaded with medicament, as discussed above. A description of the pre-loaded integrated electrode assembly and the associated electrically assisted delivery device also is found in co-pending application Ser. No. 10/820,346, filed Apr. 7, 2004, the disclosure of which is incorporated herein by reference.

Patch Fabrication Platform II—Droplet Loaded Integrated Patch

In another embodiment, unloaded gel reservoirs within an integrated patch assembly were prepared as follows to the specifications shown in Table 10:

TABLE 10

| Ingredient | % Wt. |
|---|---|
| PVP | 24.0 |
| Phenonip antimicrobial | 1.0 |
| (phenoxy ethanol and parabens) | |
| NaCl | 0.06 |
| Purified water | QS |

The gels were crosslinked by electron beam irradiation at an irradiation dose of about 2.7 Mrad (27 kGy) at an electron beam voltage of 1 MeV. The unloaded anode gel reservoirs were placed on Ag/AgCl anodes and 0.32 ml aliquots of Loading Solution A (Table 11) were placed on the reservoirs and were permitted to absorb and diffuse into the reservoir.

Patch Fabrication Platform III—Pre-Loaded Split Patch

The following components were assembled to prepare an electrode assembly, essentially as shown in the figures discussed above, for delivery of lidocaine and epinephrine by iontophoresis. The patch system used in the depth and duration studies disclosed herein was composed of two patches, an active patch (the anode) which contained lidocaine HCl, and in some cases contained epinephrine, and a return patch (the cathode) which contained only saline. Each patch was constructed of a Ag/AgCl electrode laminate, a polyvinylpyrrolidone (PVP) hydrogel reservoir, a polyethylene/acrylic adhesive laminate, and a siliconized release liner. The electrode material used for the construction of the anodes/cathodes was a Ag/AgCl printed ink material on a polyester substrate and did not exceed 5 cm².

Each drug patch was 5 cm² in active surface area with a 0.04 inch thick reservoir composed of irradiation crosslinked PVP hydrogel at a composition of 24%±1%. These hydrogels had an equilibrium capacity of greater than 2.5 (wt. of loaded hydrogel/wt. of unloaded hydrogel, gig). The 5 cm² hydrogels were laminated to silver/silver chloride ink printed electrodes. The anodes were loaded with 0.32 mL (50% by weight of the unloaded hydrogel) of lidocaine HCl and epinephrine to attain the desired compositions. For example, a 10% lidocaine HCl, 0.1% epinephrine anode would have 30% lidocaine HCl and 0.3% epinephrine in a loading solution. A 10% lidocaine HCl, 0.01% epinephrine anode would have 30% lidocaine HCl and 0.03% epinephrine in a loading solution. All anodes also had the same excipients as disclosed in Table 13 below, including anodes used in Patch Fabrication Platforms I, II, and III. The anode reservoir electrode was packaged under nitrogen in a foil laminate pouch and was stable through the entire study. Each electrode was covered with a siliconized PET release cover. The cathode always contained a saline solution, although any other fully ionized salt could have been used as the cathode was only used as the return electrode. The silver/silver chloride ink printed electrodes each had electrode capacities of more than 25 mA·min, which is enough to allow only the electrochemical reaction at the anode as Ag+Cl⁻→AgCl+e⁻, and at the cathode as AgCl+e⁻→Ag+Cl⁻. The traces leading to the controller on both electrodes were also printed in silver/silver chloride ink. The patch fabrication is described in greater detail as follows.

Backing: ethylene vinyl acetate (“EVA”) (0.10 mm (4.0 mil)±0.01 mm (0.4 mil)) coated with polyisobutylene (“PIB”) adhesive (6 mg/cm²), (Adhesive Research of Glen Rock, Pa.). The backing was dimensioned to yield a gap of between 0.94 cm (0.370 inches) and 0.95 cm (0.375 inches)±0.013 cm (0.005 inches) between the gel electrode and the outer edge of the backing at any given point on the edge of the gel. Excluding the tactile feedback notch and the wings, the tab end of the electrode had a width of 1.14 cm (0.450 inches) to 1.27 cm (0.500 inches)±0.013 cm (0.005 inches).

Tab stiffener: 7 mil PET/acrylic adhesive (Scapa Tapes of Windsor Conn.)

Printed electrode: Ag/AgCl electrode printed on DuPont 200 J102 2 mil clear printable PET film with dielectric coated Ag/AgCl traces The Ag/AgCl ink was prepared from DuPont Ag/AgCl Ink #5279, DuPont Thinner #8243, DuPont Defoamer and methyl amyl ketone (MAK). The dielectric ink was Sun Chemical Dielectric Ink # ESG56520G/S. The electrodes were printed by rotogravure substantially as shown in FIGS. 1 and 2, with a coatweight of both the electrode ink and the dielectric ink of at least about 2.6 mg/cm². The anode had a diameter of 2.26 cm (0.888 inches)±0.013 cm (0.005 inches). The cathode was essentially oval shaped, as shown in the figures. The semicircular ends of the oval both had a radius of 0.49 cm (0.193 inches)±0.013 cm (0.005 inches). The centers of the semicircular ends of the oval were separated by 1.84 cm (0.725 inches)±0.013 cm (0.005 inches).

TransferAdhesive: 6 mg/cm²+0.4 mg/cm² Ma-24A PIB transfer adhesive, (Adhesives Research). When printed onto the electrode, there was a gap of 0.076 cm (0.030 inches)±0.0076 cm (0.0030 inches) between the anode and cathode electrodes and the transfer adhesive surrounding the electrodes.

Anode Gel Reservoir: 0.10 cm (40 mil) high adhesion crosslinked polyvinylpyrrolidone (PVP) hydrogel sheet containing; 24% wt.±1% wt. PVP; 1% wt.±0.05% wt. Phenonip; 0.06% wt. NaCl to volume (QS) with purified water (USP).

The hydrogel was crosslinked by electron beam irradiation at an irradiation dose of about 2.7 Mrad (27 kGy) at an electron beam voltage of 1 MeV. The anode gel reservoir was circular, having a diameter of 2.52 cm (0.994 inches)±(0.013 cm (0.005 inches) and has a volume of about 0.8 mL (0.7 g). The reservoir was loaded by placing 334 mg of Loading Solution A onto the reservoir and allowing the solution to absorb.

Loading Solution A (anode loading solution) was prepared from the ingredients shown in Table 11, resulting in an anode reservoir composition as presented in Table 12.

TABLE 11
Loading Solution A

| Ingredient | % Wt. |
|---|---|
| Lidocaine hydrochloride | 30 |
| USP | |
| L-epinephrine bitartrate | 0.5725 |
| USP | |
| NaCl | 0.06 |
| Disodium EDTA | 0.03 |
| Citric acid | 0.06 |
| Glycerin | 30 |
| Sodium metabisulfite | 0.15 |
| Purified Water | QS |

TABLE 12
Anode Reservoir Composition

| Ingredient | mg/Reservoir | Function |
|---|---|---|
| Lidocaine HCL monohydrate, | 100 | Anesthetic |
| USP | | |
| L-epinephrine bitartrate, USP | 1.90, 1.05 | Vasoconstrictor |
| | (as free base) | |
| Glycerin | 100 | Humectant |
| Sodium Chloride | 0.52 | Anti-corrosion Agent |
| Sodium Metabisulfite | 0.5 | Antioxidant |
| Edetate Disodium | 0.1 | Chelating Agent |
| Citric Acid | 0.2 | Antioxidant Synergist, |
| | | Chelating Agent |
| Phenoxy ethanol + Parabens | 5.3 | Preservative |
| Water | 530 | Vehicle, Mobile Phase |
| PVP | 138 | Physical Structure |

Cathode Reservoir: The unloaded cathode gel consisted of a 40 mil high adhesion polyvinylpyrrolidone (PVP) hydrogel sheet containing: 24%±1% wt. PVP, 1% Phenonip antimicrobial, 0.06% wt. NaCl and purified water (Hydrogel Design Systems, Inc.). The hydrogel was crosslinked by electron beam irradiation at an irradiation dose of about 2.7 Mrad (27 kGy) at an electron beam voltage of 1 MeV. The cathode reservoir was essentially oval shaped, as shown in the figures. The semicircular ends of the oval both had a radius of 0.617 cm (0.243 inches)±0.013 cm (0.005 inches). The centers of the semicircular ends of the oval were separated by 1.84 cm (0.725 inches)±0.013 cm (0.005 inches) and the volume of the cathode reservoir was about 0.36 mL (0.37 g). The cathode reservoir was loaded by placing 227 mg of cathode loading solution onto the surface of the unloaded cathode reservoir and allowing it to fully absorb. Cathode loading Solution was prepared from the ingredients shown in Table 13, resulting in a cathode reservoir composition as presented in Table 14.

TABLE 13
Cathode Loading Solution

| Ingredient | % Wt. |
|---|---|
| Glycerin | 30 |
| NaCl | 1.28 |
| Phenoxyethanol-parabens | 0.10 |
| mixture | |
| Sodium Phosphate | 6.23 |
| monobasic | |
| Water | QS |

TABLE 14
Cathode Reservoir Composition

| Ingredient | mg/Patch | Function |
|---|---|---|
| Glycerin | 68.3 | Humectant |
| Sodium Chloride | 3 | Anti-corrosion Agent |
| Monobasic Sodium Phosphate | 14.2 | Acidulating Agent |
| Phenoxy ethanol + Parabens | 3.3 | Preservative |
| PVP | 89 | Physical Structure |
| Water | 419 | Vehicle, Mobile Phase |

Within-lot variation in solution doses and composition typically is +5%, but has not been analyzed statistically.

Release cover: 0.19 mm (7.5 ml)±0.0095 mm (0.375 mil) polyethylene terephthalate glycolate (PETG) film with silicone coating (Furon 7600 UV-curable silicon).

Nonwoven: 1.00 mm+0.2 mm Medical Nonwoven (Vilmed M1561), a blend of viscose rayon and polyester/polyethylene (PES/PE) fibers thermal bonded to PE (Freudenberg Faservliesstoffe KG Medical Nonwoven Group of Weinham, Germany).

Electrode Assembly: The electrode was assembled substantially as shown in the figures, with the anode and cathode reservoirs laminated to the electrodes. The tab stiffener was attached to the tab end of the backing of electrode assembly on the opposite side of the backing 3.8 cm (1.5 inch) long from the anode and cathode traces. The drugs were added to the unloaded anode reservoir as indicated below.

Packaging: The assembled electrode assembly was hermetically sealed in a foil-lined polyethylene pouch purged with nitrogen gas.

### EXAMPLE

### Depth and Duration of Anesthesia with Iontophoresis of Lidocaine/Eninephrine Patch

A study (n=20) was conducted to assess the depth and duration of dermal anesthesia produced by an iontophoresis drug delivery system delivering a drug formulation including 10% lidocaine anesthetic and 0.1% epinephrine vasoconstrictor and producing an approximately 5 cm² region of local anesthesia on treated skin (see the section above captioned “Depth and Duration Study” for details). The iontophoresis drug delivery system was constructed generally as shown in the attached FIGS. 2, 3, 4, 5, 5A-C, 6A-C, 7, and 7A, and as described in the above Patch Fabrication Platform I section. Each subject received two treatments to the forearms in random order: an “active” treatment (i.e., iontophoretic delivery of the lidocaine/epinephrine formulation); and a “placebo” control treatment (i.e., iontophoretic delivery of a formulation lacking lidocaine but including epinephrine) in a paired comparison design.

Pain threshold depth (PD) was measured immediately after treatment and found to average 6.37 mm, an increase of 3.28 mm from placebo treatment at the same time point. This difference is statistically significant (p<0.0001).

The depth to which all sensation was eliminated (SD) was also quantified (average 3.90 mm), and found to be increased 2.55 mm in comparison to placebo at T=0, another statistically significant difference (p<0.0001). Neither cutaneous perception of pain (CP), vascularity in the region (EI), site of treatment, nor skin thickness had a significant effect on the measurement Further, the depth of anesthesia penetration was assessed at intervals for one hour after conclusion of treatment. Pain threshold depth increased throughout the measurement period, reaching an average depth of 10.68 mm (a 7.33 mm increase from placebo), and demonstrating substantial duration of the effect. Sensory threshold depth performed consistently with PD, increasing to a maximum of 6.27 mm, a 4.93 mm increase from placebo.

No safety issues were identified during the study. Skin effects were limited to a small number of Draize 1 scores at T=60. Observed effects had resolved by the 24-hour evaluation in all cases. There was no statistical difference between the distribution of scores for active and placebo treatments; in most cases they were identical.

Whereas particular embodiments of the invention have been described herein for the purpose of illustrating the invention and not for the purpose of limiting the same, it will be appreciated by those of ordinary skill in the art that numerous variations of the details, materials and arrangement of parts may be made within the principle and scope of the invention without departing from the invention as described in the appended claims.

We claim:

1. An iontophoresis electrode assembly comprising an anode assembly including a pre-loaded hydrogel drug reservoir in electrical contact with a first electrode, the drug reservoir comprising a drug formulation including:
an anesthetic; and
a vasoconstrictor,
the electrode assembly producing clinically acceptable depth and duration of dermal anesthesia at a treated skin site on a patient.

2. The electrode assembly of claim 1, wherein the average depth to which all sensation is eliminated on advancing an 18 gauge needle into the treated skin of a forearm of a patient immediately after treatment with the electrode assembly and the drug formulation is greater than 4 mm.

3. The electrode assembly of claim 1, wherein the average depth to which all sensation is eliminated on advancing an 18 gauge needle into the treated skin of a forearm of a patient immediately after treatment with the electrode assembly and the drug formulation is greater than 5 mm.

4. The electrode assembly of claim 1, wherein the average depth to which all sensation is eliminated on advancing an 18 gauge needle into the treated skin of the forearm of a patient immediately after treatment with the electrode assembly and the drug formulation is at least 6 mm.

5. The electrode assembly of claim 1, wherein the average depth to which all sensation is eliminated on advancing an 18 gauge needle into the treated skin of a forearm of a patient immediately after treatment with the electrode assembly and the drug formulation is statistically significantly greater (p<0.0001) than the average depth to which sensation of pain is eliminated assessed in an identical manner on a substantially equivalent skin site immediately after treatment using an identical electrode assembly iontophoretically delivering a placebo.

6. The electrode assembly of claim 1, wherein the average depth to which all sensation is eliminated on advancing an 18 gauge needle into the treated skin of a forearm of a patient immediately after treatment with the electrode assembly and the drug formulation is at least 2 mm greater than the average depth to which all sensation is eliminated assessed in an identical manner on a substantially equivalent skin site immediately after treatment using an identical electrode assembly iontophoretically delivering a placebo.

7. The electrode assembly of claim 1, wherein the average depth to which all sensation is eliminated on advancing an 18 gauge needle into the treated skin of a forearm of a patient after treatment with the electrode assembly and the drug formulation does not decrease within the first hour immediately after ending the treatment.

8. The electrode assembly of claim 1, wherein the average pain threshold depth on advancing an 18 gauge needle into the treated skin of a forearm of a patient immediately after treatment with the electrode assembly and the drug formulation is greater than 2 mm.

9. The electrode assembly of claim 1, wherein the average pain threshold depth on advancing an 18 gauge needle into the treated skin of a forearm of a patient immediately after treatment with the electrode assembly and the drug formulation is at least 3 mm.

10. The electrode assembly of claim 1, wherein the average pain threshold depth on advancing an 18 gauge needle into the treated skin of a forearm of a patient immediately after treatment with the electrode assembly and the drug formulation is statistically significantly greater (p<0.0001) than the average pain threshold depth assessed in an identical manner on a substantially equivalent skin site immediately after treatment using an identical electrode assembly iontophoretically delivering a placebo.

11. The electrode assembly of claim 1, wherein the average pain threshold depth on advancing an 18 gauge needle into the treated skin of a forearm of a patient immediately after treatment with the electrode assembly and the drug formulation is at least 2 mm greater than the average pain threshold depth assessed in an identical manner on a substantially equivalent skin site immediately after treatment using an identical electrode assembly iontophoretically delivering a placebo.

12. The electrode assembly of claim 1, wherein the average pain threshold depth on advancing an 18 gauge needle into the treated skin of a forearm of a patient immediately after treatment with the electrode assembly and the drug formulation is at least 3 mm greater than the average pain threshold depth assessed in an identical manner on a substantially equivalent skin site immediately after treatment using an identical electrode assembly iontophoretically delivering a placebo.

13. The electrode assembly of claim 1, wherein the average pain threshold on advancing an 18 gauge needle into the treated skin of a forearm of a patient after treatment with the electrode assembly and the drug formulation does not decrease within the first hour immediately after ending the treatment.

14. The electrode assembly of claim 1, wherein the anesthetic is lidocaine.

15. The electrode assembly of claim 1, wherein the vasoconstrictor is epinephrine.

16. The electrode assembly of claim 1, wherein the anesthetic is lidocaine and the vasoconstrictor is epinephrine.

17. The electrode assembly of claim 16 wherein the average depth to which all sensation is eliminated on advancing an 18 gauge needle into the treated skin of a forearm of a patient immediately after treatment with the electrode assembly and the drug formulation is greater than 4 mm.

18. The electrode assembly of claim 16 wherein the average depth to which all sensation is eliminated on advancing an 18 gauge needle into the treated skin of a forearm of a patient immediately after treatment with the electrode assembly and the drug formulation is greater than 5 mm.

19. The electrode assembly of claim 16, wherein the average depth to which all sensation is eliminated on advancing an 18 gauge needle into the treated skin of the forearm of a patient immediately after treatment with the electrode assembly and the drug formulation is at least 6 mm.

20. The electrode assembly of claim 16, wherein the average depth to which all sensation is eliminated on advancing an 18 gauge needle into the treated skin of a forearm of a patient immediately after treatment with the electrode assembly and the drug formulation is statistically significantly greater (p<0.0001) than the average depth to which sensation of pain is eliminated assessed in an identical manner on a substantially equivalent skin site immediately after treatment using an identical electrode assembly iontophoretically delivering a placebo.

21. The electrode assembly of claim 16, wherein the average depth to which all sensation is eliminated on advancing an 18 gauge needle into the treated skin of a forearm of a patient immediately after treatment with the electrode assembly and the drug formulation is at least 2 mm greater than the average depth to which all sensation is eliminated assessed in an identical manner on a substantially equivalent skin site immediately after treatment using an identical electrode assembly iontophoretically delivering a placebo.

22. The electrode assembly of claim 16, wherein the average depth to which all sensation is eliminated on advancing an 18 gauge needle into the treated skin of a forearm of a patient immediately after treatment with the electrode assembly and the drug formulation is at least 3 mm greater than the average depth to which all sensation is eliminated assessed in an identical manner on a substantially equivalent skin site immediately after treatment using an identical electrode assembly iontophoretically delivering a placebo.

23. The electrode assembly of claim 16, wherein the average depth to which all sensation is eliminated on advancing an 18 gauge needle into the treated skin of a forearm of a patient after treatment with the electrode assembly and the drug formulation does not decrease within the first hour immediately after ending the treatment.

24. The electrode assembly of claim 16, wherein the average pain threshold depth on advancing an 18 gauge needle into the treated skin of a forearm of a patient immediately after treatment with the electrode assembly and the drug formulation is greater than 2 mm.

25. The electrode assembly of claim 16, wherein the average pain threshold depth on advancing an 18 gauge needle into the treated skin of a forearm of a patient immediately after treatment with the electrode assembly and the drug formulation is greater than 3 mm.

26. The electrode assembly of claim 16, wherein the average pain threshold depth on advancing an 18 gauge needle into the treated skin of a forearm of a patient immediately after treatment with the electrode assembly and the drug formulation is statistically significantly greater (p<0.0001) than the average pain threshold depth assessed in an identical manner on a substantially equivalent skin site immediately after treatment using an identical electrode assembly iontophoretically delivering a placebo.

27. The electrode assembly of claim 16, wherein the average pain threshold depth on advancing an 18 gauge needle into the treated skin of a forearm of a patient immediately after treatment with the electrode assembly and the drug formulation is at least 2 mm greater than the average pain threshold depth assessed in an identical manner on a substantially equivalent skin site immediately after treatment using an identical electrode assembly iontophoretically delivering a placebo.

28. The electrode assembly of claim 16, wherein the average pain threshold depth on advancing an 18 gauge needle into the treated skin of a forearm of a patient immediately after treatment with the electrode assembly and the drug formulation is at least 3 mm greater than the average pain threshold depth assessed in an identical manner on a substantially equivalent skin site immediately after treatment using an identical electrode assembly iontophoretically delivering a placebo.

29. The electrode assembly of claim 16, wherein the average pain threshold on advancing an 18 gauge needle into the treated skin of a forearm of a patient after treatment with the electrode assembly and the drug formulation does not decrease within the first hour immediately after ending the treatment.

30. The electrode assembly of claim 1, wherein the drug reservoir comprises lidocaine in an amount greater than about 2% by weight of the reservoir.

31. The electrode assembly of claim 1, wherein the drug reservoir comprises lidocaine in an amount of about 10% by weight of the reservoir.

32. The electrode assembly of claim 1, wherein the drug reservoir comprises epinephrine in an amount greater than about 0.005% by weight of the reservoir.

33. The electrode assembly of claim 1, wherein the drug reservoir comprises epinephrine in an amount greater than about 0.01% by weight of the reservoir.

34. The electrode assembly of claim 1, wherein the drug reservoir comprises epinephrine in an amount between about 0.01% by weight of the reservoir and 0.3% by weight of the reservoir.

35. The electrode assembly of claim 1, wherein the drug reservoir comprises epinephrine in an amount of about 0.1% by weight of the reservoir.

36. The electrode assembly of claim 1, wherein the drug reservoir comprises epinephrine in an amount between about 0.01% by weight of the reservoir and about 0.3% by weight of the reservoir, and lidocaine in an amount greater than about 2% by weight of the reservoir.

37. The electrode assembly of claim 1, wherein the drug reservoir further comprises an alkaline metal halide salt in an amount between about 0.001% by weight of the reservoir and 1.0% by weight of the reservoir.

38. The electrode assembly of claim 1, wherein the drug reservoir comprises an alkaline metal halide salt in an amount of about 0.06% by weight of the reservoir.

39. The electrode assembly of claim 38, wherein the alkaline metal halide salt is sodium chloride.

40. The electrode assembly of claim 1, further comprising a cathode assembly comprising a second electrode and a return hydrogel in electrical contact with the second electrode, wherein the first electrode and the second electrode are attached to a backing.

41. The electrode assembly of claim 40, further comprising an electrically conductive anode trace attached to the backing and electrically connected to the first electrode, and an electrically conductive cathode trace attached to the backing and electrically connected to the second electrode.

42. The electrode assembly of claim 40, wherein the first and second electrodes and the anode and cathode traces comprise silver/silver chloride-containing ink.

43. The electrode assembly of claim 40, wherein the first electrode and the second electrode are silver/silver chloride electrodes.

44. The electrode assembly of claim 1, wherein the drug reservoir comprises a hydrogel.

45. The electrode assembly of claim 44, wherein the hydrogel is polyvinyl pyrrolidone.

46. The electrode assembly of claim 44, wherein the hydrogel is from about 15% to about 17% by weight polyvinyl pyrrolidone.

47. The electrode assembly of claim 1, wherein the reservoir comprises from about 2% by weight to about 12% by weight lidocaine, and from about 0.001% by weight to about 0.3% by weight epinephrine.

48. The electrode assembly of claim 47, wherein the reservoir has a volume of about 1 milliliter and comprises about 100 milligrams lidocaine HCl and about 1.05 milligrams epinephrine bitartrate.

49. The electrode assembly of claim 1, wherein the unloaded drug reservoir has a thickness ranging from 0.89 mm to about 1.14 mm.

50. The electrode assembly of claim 1, wherein the drug reservoir comprises lidocaine HCl and epinephrine bitartrate in a mass ratio of about 50:1 to about 1000:1.

51. The electrode assembly of claim 1, wherein the drug reservoir comprises lidocaine HCl and epinephrine bitartrate in a mass ratio of about 70:1 to about 125:1.

52. The electrode assembly of claim 16, wherein the reservoir further comprises one or more of parabens, sodium metabisulfite, a chelating agent, citric acid, glycerin and sodium chloride.

53. The electrode assembly of claim 16, wherein the anode assembly is prepared by contacting an unloaded reservoir including from about 0.001% by weight to about 1.0% by weight sodium chloride with a solution including the lidocaine and the epinephrine.

54. A method of producing local anesthesia in a patient, comprising:
applying a charge density of at least about 1.5 mA·min/cm² for at least about 5 minutes to an electrically assisted drug delivery system comprising an anode assembly including a reservoir in electrical contact with the patient, wherein the reservoir is pre-loaded with a drug formulation including an anesthetic and a vasoconstrictor, the electrically assisted drug delivery system producing clinically acceptable depth and duration of dermal anesthesia at a treated site.

55. The method of claim 54, wherein the average depth to which all sensation is eliminated on advancing an 18 gauge needle into the treated skin of a forearm of a patient immediately after treatment with the electrode assembly and the drug formulation is greater than 4 mm.

56. The method of claim 54, wherein the average depth to which all sensation is eliminated on advancing an 18 gauge needle into the treated skin of a forearm of a patient immediately after treatment with the electrode assembly and the drug formulation is greater than 5 mm.

57. The method of claim 54, wherein the average depth to which all sensation is eliminated on advancing an 18 gauge needle into the treated skin of a forearm of a patient immediately after treatment with the electrode assembly and the drug formulation is greater than 6 mm.

58. The method of claim 54, wherein the average depth to which all sensation is eliminated on advancing an 18 gauge needle into the treated skin of a forearm of a patient immediately after treatment with the electrode assembly and the drug formulation is statistically significantly greater (p<0.0001) than the average depth to which sensation of pain is eliminated assessed in an identical manner on a substantially equivalent skin site immediately after treatment using an identical electrode assembly iontophoretically delivering a placebo.

59. The method of claim 54, wherein the average depth to which all sensation is eliminated on advancing an 18 gauge needle into the treated skin of a forearm of a patient immediately after treatment with the electrode assembly and the drug formulation is at least 2 mm greater than the average depth to which all sensation is eliminated assessed in an identical manner on a substantially equivalent skin site immediately after treatment using an identical electrode assembly iontophoretically delivering a placebo.

60. The method of claim 54, wherein the average depth to which all sensation is eliminated on advancing an 18 gauge needle into the treated skin of a forearm of a patient after treatment with the electrode assembly and the drug formulation does not decrease within the first hour immediately after ending the treatment.

61. The method of claim 54, wherein the average pain threshold depth on advancing an 18 gauge needle into the treated skin of a forearm of a patient immediately after treatment with the electrode assembly and the drug formulation is greater than 2 mm.

62. The method of claim 54, wherein the average pain threshold depth on advancing an 18 gauge needle into the treated skin of a forearm of a patient immediately after treatment with the electrode assembly and the drug formulation is at least 3 mm.

63. The method of claim 54, wherein the average pain threshold depth on advancing an 18 gauge needle into the treated skin of a forearm of a patient immediately after treatment with the electrode assembly and the drug formulation is statistically significantly greater (p<0.0001) than the average pain threshold depth assessed in an identical manner on a substantially equivalent skin site immediately after treatment using an identical electrode assembly iontophoretically delivering a placebo.

64. The method of claim 54, wherein the average pain threshold depth on advancing an 18 gauge needle into the treated skin of a forearm of a patient immediately after treatment with the electrode assembly and the drug formulation is at least 2 mm greater than the average pain threshold depth assessed in an identical manner on a substantially equivalent skin site immediately after treatment using an identical electrode assembly iontophoretically delivering a placebo.

65. The method of claim 54, wherein the average pain threshold depth on advancing an 18 gauge needle into the treated skin of a forearm of a patient immediately after treatment with the electrode assembly and the drug formulation is at least 3 mm greater than the average pain threshold depth assessed in an identical manner on a substantially equivalent skin site immediately after treatment using an identical electrode assembly iontophoretically delivering a placebo.

66. The method of claim 54, wherein the average pain threshold on advancing an 18 gauge needle into the treated skin of a forearm of a patient after treatment with the electrode assembly and the drug formulation does not decrease within the first hour immediately after ending the treatment.

67. The method of claim 54, wherein the anesthetic is lidocaine and the vasoconstrictor is epinephrine.

68. The method of claim 67, wherein the average depth to which all sensation is eliminated on advancing an 18 gauge needle into the treated skin of a forearm of a patient immediately after treatment with the electrode assembly and the drug formulation is greater than 4 mm.

69. The method of claim 67, wherein the average depth to which all sensation is eliminated on advancing an 18 gauge needle into the treated skin of a forearm of a patient immediately after treatment with the electrode assembly and the drug formulation is greater than 5 mm.

70. The method of claim 67, wherein the average depth to which all sensation is eliminated on advancing an 18 gauge needle into the treated skin of a forearm of a patient immediately after treatment with the electrode assembly and the drug formulation is at least 6 mm.

71. The method of claim 67, wherein the average depth to which all sensation is eliminated on advancing an 18 gauge needle into the treated skin of a forearm of a patient immediately after treatment with the electrode assembly and the drug formulation is statistically significantly greater (p<0.0001) than the average depth to which sensation of pain is eliminated assessed in an identical manner on a substantially equivalent skin site immediately after treatment using an identical electrode assembly iontophoretically delivering a placebo.

72. The method of claim 67, wherein the average depth to which all sensation is eliminated on advancing an 18 gauge needle into the treated skin of a forearm of a patient after treatment with the electrode assembly and the drug formulation does not decrease within the first hour immediately after ending the treatment.

73. The method of claim 67 wherein the average pain threshold depth on advancing an 18 gauge needle into the treated skin of a forearm of a patient immediately after treatment with the electrode assembly and the drug formulation is greater than 2 mm.

74. The method of claim 67, wherein the average pain threshold depth on advancing an 18 gauge needle into the treated skin of a forearm of a patient immediately after treatment with the electrode assembly and the drug formulation is greater than 3 mm.

75. The method of claim 67, wherein the average pain threshold depth on advancing an 18 gauge needle into the treated skin of a forearm of a patient immediately after treatment with the electrode assembly and the drug formulation is statistically significantly greater (p<0.0001) than the average pain threshold depth assessed in an identical manner on a substantially equivalent skin site immediately after treatment using an identical electrode assembly iontophoretically delivering a placebo.

76. The method of claim 67, wherein the average pain threshold depth on advancing an 18 gauge needle into the treated skin of a forearm of a patient immediately after treatment with the electrode assembly and the drug formulation is at least 2 mm greater than the average pain threshold depth assessed in an identical manner on a substantially equivalent skin site immediately after treatment using an identical electrode assembly iontophoretically delivering a placebo.

77. The method of claim 67, wherein the average pain threshold depth on advancing an 18 gauge needle into the treated skin of a forearm of a patient immediately after treatment with the electrode assembly and the drug formulation is at least 3 mm greater than the average pain threshold depth assessed in an identical manner on a substantially equivalent skin site immediately after treatment using an identical electrode assembly iontophoretically delivering a placebo.

78. The method of claim 67, wherein the depth to which all sensation is eliminated on advancing an 18 gauge needle into the treated skin of a forearm of a patient immediately after treatment with the electrode assembly and the drug formulation is at least 2 mm greater than the average pain threshold depth assessed in an identical manner on a substantially equivalent skin site immediately after treatment using an identical electrode assembly iontophoretically delivering a placebo.

79. The method of claim 67, wherein the average pain threshold on advancing an 18 gauge needle into the treated skin of a forearm of a patient after treatment with the electrode assembly and the drug formulation does not decrease within the first hour immediately after ending the treatment.

80. The method of claim 54, wherein the charge density is between about 1.5 mA·min/cm² and about 4.2 mA·min/cm².

81. The method of claim 54, wherein the charge density is about 3.4 mA·min/cm².

82. The method of claim 54, wherein the charge density is applied for from about 5 to about 20 minutes.
